(19) United States
(12) Patent Application Publication
(10) Pub. No.: US 2009/0010983 A1
(43) Pub. Date: Jan. 8, 2009

(54) Alginate Coated, Polysaccharide Gel-Containing Foam Composite, Preparative Methods, and Uses Thereof

(75) Inventors: Jan Egil Melvik, Oslo (NO); Therese Andersen, Sande i Vestfold (NO)

Correspondence Address: Patent Administrator;FMC CORPORATION, 1735 Market Street, Philadelphia, PA, 19103, US

(73) Assignee: FMC Corporation, Philadelphia, PA (US)

(21) Appl. No.: 12/138,738

(22) Filed: Jun. 13, 2008

Related U.S. Application Data
(60) Provisional application No. 60/943,797, filed on Jun. 13, 2007.

(51) Int. Cl. A61K 35/12; C12N 5/06; A61F 2/04; A61P 3/10; A61K 47/36

(52) U.S. Cl. 424/422; 435/402; 435/375; 435/325; 514/777; 424/93.7

(57) ABSTRACT

The invention relates to composites comprising a polysaccharide gelled within pores of a foam and an polysaccharide coating, methods of preparation, and uses thereof, for example, in biomedical applications such as cell culture media and implants, controlled release delivery systems, food applications, industrial applications, and personal care applications such as cosmetic and oral hygiene.

### FIELD OF THE INVENTION

The invention relates to a composite comprising a polysaccharide gelled within pores of a foam and a coating of polysaccharide, methods of preparation of the composite, and uses thereof. This composite is particularly useful in biomedical applications for example cell culture media and implants, controlled release delivery systems, food applications, industrial applications, and personal care applications including cosmetic and oral hygiene.

### BACKGROUND OF THE INVENTION

A need exists to provide a product suitable for use for example in wound management, tissue engineering, tissue regeneration and cell immobilization. This invention is directed to this need and others.

### SUMMARY OF THE INVENTION

In one aspect, the present invention provides composites comprising:
i) a foam having pores, wherein said foam comprises gel-forming ions distributed through at least a part of the foam;
ii) a gel comprising a polysaccharide with gelling sites, wherein the gel is located within the pores of the foam and interacts with the foam; and
iii) a coating comprising a polysaccharide with gelling sites and gel-forming ions, wherein said coating completely covers the outer surface of said foam.

In another aspect, the present invention provides composites further comprising cells within the gel that is in the pores of the foam of the composite.

In another aspect, the present invention further provides methods of producing any of the composites of the invention, including the various composite embodiments described herein and combinations of those embodiments, comprising:
i) contacting a first liquid component with a foam having pores, wherein said first liquid component comprises a polysaccharide having gelling sites capable of forming a gel upon contact with said ions, whereby said gel forms upon contact with the said gel-forming ions; and
ii) forming said coating on the outer surface of said foam;
   wherein:

said foam comprises a polymer and gel-forming ions, wherein said ions are distributed through at least a part of the foam; and

said coating comprises a polysaccharide with gelling sites and gel-forming ions, wherein said coating completely covers the outer surface of said foam.

In a further aspect, the invention provides for the methods and use of a composite produced according to the method of the invention. The composite is particularly suitable for use in medical applications, for example in wound management, tissue engineering and tissue regeneration and cell immobilization.

The present invention provides a method of administering one or more composites to a patient in need thereof, comprising implanting said one or more composites in said patient.

The present invention further provides a composite described herein for use in a method of treatment of the human or animal body by therapy. In some embodiments, the composite is used in a method of treatment of diabetes or reducing blood glucose levels.

The present invention further provides a kit for implanting one or more composites in a patient in need thereof, comprising one or more composites of the invention.

The present invention further provides a method for promoting cell proliferation in the composites described herein comprising washing the composite with a solution, adjusted for osmotic pressure neutral to living cells, and which solution contains gel-forming ions.

The present invention further provides a method for inhibiting cell proliferation comprising forming a composite by the methods of the invention, wherein the liquid component comprises cells, said polysaccharide being gelled within the pores of the foam wherein the gel-forming ion comprises strontium ion.

The present invention further provides a method for recovery of cells from the composites described herein comprising contacting an aqueous solution comprising a recovery agent with the gel comprising the soluble polysaccharide wherein the said aqueous solution is adjusted to provide an osmotic pressure neutral to living cells.

The present invention further provides a method of promoting cell growth comprising implanting a composite described herein, wherein the foam comprises alginate into a human or animal body for the purpose of establishing cell growth.

The present invention further provides a method for preventing adhesion of tissue to adjacent tissue comprising applying to the tissue a composite described herein such that it provides a barrier between the tissue and the adjacent tissue.

The present invention further provides the use of the composites described herein as matrices for cell immobilization and/or proliferation for an in vitro tissue culture application or an in vivo tissue engineering application.

### BRIEF DESCRIPTION OF THE FIGURES

FIG. 1 depicts a representation of an embodiment of the foam composite, wherein cells are part of the gel.

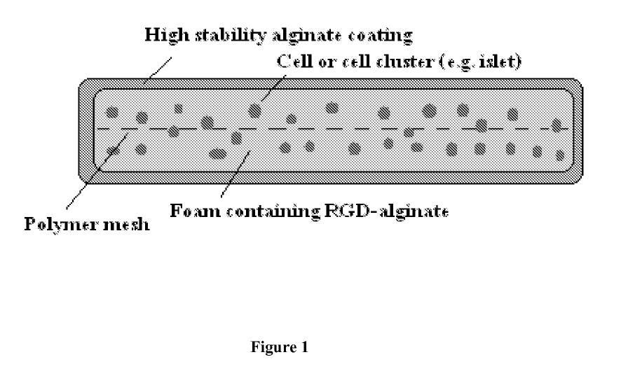

### DETAILED DESCRIPTION OF THE INVENTION

### Definitions

As used herein, the term “about” means plus or minus 10% of the value.

As used herein, the term “alginate” refers to salts of alginic acid, and modified alginates. Alginic acid, which is isolated from seaweed, is a polyuronic acid made up of two uronic acids: D-mannuronic acid and L-guluronic acid. The ratio of mannuronic acid and guluronic acid varies with factors such as seaweed species, plant age, and part of the seaweed (e.g., stem, leaf). Alginic acid is substantially insoluble in water. It forms water-soluble salts with alkali metals, such as sodium, potassium, and, lithium; magnesium; ammonium; and the substituted ammonium cations derived from lower amines, such as methyl amine, ethanol amine, diethanol amine, and triethanol amine. The salts are soluble in aqueous media above pH 4, but are converted to alginic acid when the pH is lowered below about pH 4. A thermo-irreversible water-insoluble alginate gel is formed in the presence of gel-forming ions, e.g. calcium, barium, strontium, zinc, copper(+2), aluminum, and mixtures thereof, at appropriate concentrations. The alginate gels can be solubilized by soaking in a solution of soluble cations or chelating agents for the gel-forming ions, for example EDTA, citrate and the like.

Water-insoluble alginate salts, in which the principal cation is calcium, are found in the fronds and stems of seaweeds of the class Phaeophyceae, examples of which are Fucus vesiculosus, Fucus spiralis, Ascophyllum nodosum, Macrocystis pyrifera, Alaria esculenta, Eclonia maxima, Lessonia nigrescens, Lessonia trabeculata, Laminaria japonica, Durvillea antarctica, Laminaria hyperborea, Laminaria longicruris, Laminaria digitata, Laminaria saccharina, Laminaria cloustoni, and Saragassum sp. Methods for the recovery of alginic acid and its water-soluble salts, especially sodium alginate, from natural sources are well known, and are described, for example, in Green, U.S. Pat. No. 2,036,934, and Le Gloahec, U.S. Pat. No. 2,128,551. Suitable alginates include, but are not limited to, the PRONOVA UP and SLM series (NovaMatrix, FMC Corp., Oslo, Norway).

As used herein, “chitosan” refers to a linear polysaccharide comprising β-(1→4)-linked 2-acetamido-2-dexoy-D-glucopyranose (GlcNAc) and 2-amino-2-deoxy-D-glucopyranose (GlcN), and modified chitosans. Chitosan is N-deacetylated derivative of chitin, which consists nearly entirely of β-(1→4)-linked 2-acetamido-2-dexoy-D-glucopyranose (GlcNAc). Commercially chitosan is made by alkaline N-deacetylation of chitin. The heterogeneous deacetylation process combined with removal of insoluble compound results in a chitosan product which possesses a random distribution of GlcNAc and GlcN-units along the polymer chain. The amino group in chitosan has an apparent pKₐ-value of about 6.5 and at a pH below this value, the free amino group will be protonized so the chitosan salt dissolved in solution will carry a positive charge. Accordingly, chitosan is able to react with negatively charged components it being a direct function of the positive charge density of chitosan.

Advantageously, the cationic nature of chitosan provides a bioadhesive property. In addition, chitosan may precipitate red blood cells due to their negative charge providing benefits in forming blood clots and in reducing the level of fibrin during healing so reducing the formation of scar tissue. Chitosan may be degraded by lysozyme and other related enzymes occurring in a mammalian body, for example the human body. In use the chitosan in a foam of the present invention will suitably be degraded by lysozyme found in mammals in saliva, tears, blood serum and in interstitial fluid. A composite having a chitosan foam may advantageously be employed in wound management, as a bioadhesive and in other applications in the human or animal body. The degradation products of chitosan are glucosamine and N-acetylglucosamine which are non-toxic in mammals. The rate of biodegradation of implanted chitosan foams by the lysozyme can be modified by varying the degree of chitosan deacetylation since acetylation protects the polymer from enzymatic degradation. Chitosans with higher degrees of deacetylation are also more resistant to random depolymerization by acid hydrolysis due to a protective effect of the positive charge.

Chitosans suitable for use in the present invention may be in the form of the chitosan base, a water-soluble chitosan salt or a modified chitosan. Chitosan base may require dilute acid to dissolve for example 1 wt % acetic acid. Chitosan is soluble in aqueous media at acidic pH, where the polysaccharide will be highly positively charged. High molecular weight chitosans with a random distribution of monomer units and a degree of deacetylation (DA) between 40% and 60% are soluble at neutral pH. Chitosans shown increasing solubility at higher pH-values with decreasing DA. Also, by depolymerising chitosans with DA above 60%, their water solubility at neutral pH-values can be increased.

Generally, chitosan requires an acidic environment for dissolution. By dissolving chitosan in an appropriate acid, the chitosan salt is obtained upon drying. Suitable chitosan salts include chitosan chloride, chitosan glutamate, chitosan lactate, chitosan maleate, chitosan malate, chitosan malonate, chitosan succinate, chitosan formate, chitosan aspartate, chitosan acetate, chitosan propionate, chitosan nitrate, chitosan nicotinate, and chitosan adipate. For example, chitosan glutamate is chitosan converted into the glutamate salt form by dissolving chitosan in glutamic acid. Glutamic acid is present at a stoichiometric amount to the number of GlcN units. Chitosan chloride contains a stoichiometric amount of hydrochloride to the number of GlcN units.

Salts of chitosan are generally soluble water, and the pH of a 1% solution of chitosan salt is typically between 4 and 6. The functional properties of chitosan are influenced by the degree of deacetylation and molecular weight and molecular weight distribution. Suitably, the degree of acetylation ranges from 40% to 100%, preferably 50% to 100%. In some embodiments, the degree of acetylation is preferably 80 to 99%, more preferably 80 to 95%. Suitable molecular weights are in the range 10 kDa to 1000 kDa.

Suitable modified chitosans contain moieties covalently linked to the chitosan for example peptide coupled chitosan. Modified chitosans can be tailored by selection of moieties and their concentration in the modified chitosan to add, modify or alter properties or functionalities of the chitosan such as crosslinking capability, solubility, rate of biodegradability of the ability to bind, for example, specific cells, pharmaceuticals or peptides.

Enzymatic degradation allows the foam to be designed in such a manner that the product may perform its function and then be removed from the body through degradation. Suitable chitosans include, but are not limited to, the Protasan series (NovaMatrix, FMC Corp., Oslo, Norway).

As used herein, “pectin” is a naturally occurring polysaccharide found in the roots, stems, leaves, and fruits of various plants, especially the peel of citrus fruits such as limes, lemons, grapefruits, and oranges. Pectins contain polymeric units derived from D-galacturonic acid. Commercial products include high methoxy pectin and low methoxy pectin (and derivatives such as amidated pectins) in which 20-60% of the units derived from D-galacturonic acid, depending on the source of the pectin, are esterified with methyl groups. Pectate (pectinate) is fully de-esterified pectin with up to 20% of the units derived from D-galacturonic acid.

As used herein, “carrageenan” refers to a group of sulfated galactans which may be extracted from red seaweeds. Carrageenans are linear chains of D-galactopyranosyl units joined with alternating (1→3) α-D and (1→4) β-D-glycosidic linkages. Carrageenans may, in part, be distinguished by the degree and position of sulfation. Most sugar units have one or two sulfate groups esterified to a hydroxyl group at carbons C-2 or C-6. Suitable carrageenans include kappa carrageenan, iota carrageenan, and kappa II carrageenan and blends thereof. Sodium carrageenans are soluble at room temperature. Carrageenans can be prepared with low contents of gel-forming ions by known techniques. Carrageenan gels are thermoreversible. Higher levels of gel-forming ions may increase the temperature at which the gel can melted. Kappa carrageenans produce strong rigid gels while iota carrageenans are elastic and compliant. Kappa II carrageenans which are copolymers of kappa and iota form weak gels. Gel-forming ions for specific carrageenans are known in the art and include potassium and calcium. Lambda carrageenans do not form gels in water but may be useful in blends, for example, to modify the mechanical properties of the resulting gel. A preferred carrageenan is iota carrageenan. Iota carrageenan has a repeating unit of D-galactose-4-sulfate-3,6-anhydro-D-galactose-2-sulfate providing a sulfate ester content of about 25 to 34%.

As used herein “hyaluronic acid” refers to hyaluronic acid (HA), salts thereof and modified hyaluronates. Sodium hyaluronate is an abundant glycosaminoglycan found in the extracellular matrix of skin, joints, eyes and most organs and tissues of all higher animals. Non animal derived HA may be fermented from Streptococcus zooepidemicus Hyaluronic acid from a non-animal source is preferred for use in the present invention. Hyaluronic acid is a linear copolymer composed of (β-1,4)-linked D-glucuronate (D) and (β-1,3)-N-acetyl-D-glucosamine (N). The coiled structure of hyaluronate can trap approximately 1000 times its weight in water. These characteristics give the molecule advantageous physicochemical properties as well as distinct biological functions and is desirable for use as a building block for biocompatible and biointeractive materials in pharmaceutical delivery, tissue engineering and viscosupplementation.

Hyaluronic acid or hyaluronate is a natural component in mammalian organisms and is enzymatically biodegradable by hyaluronidases. The half-life of hyaluronate in endothelial tissue is less than a day, and the natural turnover of the polymer in adults is approximately 7 g a day. A mild to moderate covalent modification of hyaluronan will increase the in vivo stability and retention time from days up to months or a year.

Suitable modified hyaluronates include those containing moieties covalently linked to the hyaluronates and may include for example peptide coupled hyaluronates. A preferred modified hyaluronate suitably has a covalently modified carboxyl group and/or hydroxyl group on the D and N monomer units respectively. Modified hyaluronates can be tailored by selection of moieties and their concentration in the modified hyaluronates to add, modify or alter properties or functionalities of the hyaluronates such as crosslinking capability, solubility, rate of biodegradability of the ability to bind, for example, specific cells, drugs or peptides.

Hyaluronic acid is thought to play an important role in the early stages of connective tissue healing and scarless fetal wound healing and regulate cell mobility, adhesion, and proliferation and is especially useful in tissue engineering and tissue regeneration applications.

As used herein, the term “interact” means that the gel is in physical contact with the foam, for example, by being retained in the pores of the foam by physical interlocking of the gel and foam. Alternatively, the interaction may be chemical, e.g., through bonding between the foam and the polysaccharide by means of the gel-forming ions forming “bridges”.

As used herein, the term “located” means that the gel is substantially located in the pores of the foam, e.g, greater than 50%, 60%, 70%, 80%, 90%, 95%, 96%, 97%, 98%, 99%, or 99.9% of said gel is located in the pores of the foam.

As used herein, the term “distributed through at least a portion of the foam” means that the gel-forming ions are present in a part of the foam in a sufficient quantity to form a gel with at least a portion of the polysaccharide in the gel. Preferably, the gel-forming ions are substantially and uniformly distributed. Preferably the gel-forming ions are present in at least some of the internal pores of the foam rather than only surface pores. The gel-forming ions are preferably substantially evenly distributed in the foam.

As used herein, the term “gelling sites” refers to functional groups on the polysaccharide of the gel, coating, or first liquid component that can interact with the gel-forming ions through ionic bonding to facilitate the formation of a gel. For example, an alginate has gelling sites which are carboxylate groups which can interact with gel-forming ions such calcium ions.

As used herein, the term “modified alginate” includes alginates covalently linked to organic moieties or peptide. For example, alginate may be reacted with an organic moiety like alkylene oxide, such as ethylene oxide or propylene oxide, to form a glycol alginate. The glycol is bonded to the alginate through the carboxyl groups. Typically, alginate is reacted with propylene oxide to form propylene glycol alginate (PGA). Preparation of propylene glycol alginate is disclosed in Strong, U.S. Pat. No. 3,948,881, Pettitt, U.S. Pat. No. 3,772,266, and Steiner, U.S. Pat. No. 2,426,125. Preferably, the propylene glycol alginate has a degree of esterification of about 40% to about 95%, more preferably about 70% to 95%. Mixtures of propylene glycol alginates of different molecular weights may also be used. Aluminum ions are suitable for gelling glycol alginates.

As used herein, the term “cell adhesion sequence modified polysaccharide” means a polysaccharide covalently bound to at least one peptide comprising one or more cell adhesion sequences. For example peptide-coupled polysaccharides are prepared by means known in the art. For example, modified alginates are disclosed in U.S. Pat. No. 6,642,363 (Mooney). Peptide-coupled polysaccharides are preferred for use for example in immobilizing cells to promote cell proliferation and cell differentiation. Peptide-coupled polysaccharides are preferably employed in combination with non-modified polysaccharides.

U.S. Pat. Nos. 4,988,621, 4,792,525, 5,965,997, 4,879,237, 4,789,734 and 6,642,363, which are incorporated herein by reference, disclose numerous examples of cell adhesion sequence peptides. Suitable peptides include, but are not limited to, peptides having about 10 amino acids or less. In some embodiments, cell attachment peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:1), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). In some embodiments, cell attachment peptides comprise RGD, YIGSR (SEQ ID NO: 1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO: 13), VTCG (SEQ ID NO: 14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22) and further comprise additional amino acids, such as for example, 1-10 additional amino acids, including but not limited 1-10 G residues at the N or C terminal. For example, a suitable peptide may have the formula (Xaa)ₙ—SEQ-(Xaa)ₙ wherein Xaa are each independently any amino acid, n=0-7 and SEQ=a peptide sequence selected from the group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22, and the total number of amino acids is less than 22, preferably less that 20, preferably less that 18, preferably less that 16, preferably less that 14, preferably less that 12, preferably less that 10. In some embodiments, cell attachment peptides consist of RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). Biologically active molecules for cell adhesion or other cellular interaction may include EGF, VEGF, b-FGF, FGF, TGF, TGF-β or proteoglycans. Cell attachment peptides comprising RGD may be in some embodiments, 3, 4, 5, 6, 7, 8, 9 or 10 amino acids in length. Examples include, but are not limited to, RGD, GRGDS (SEQ ID NO:6), RGDV (SEQ ID NO:7), RGDS (SEQ ID NO:11), RGDF (SEQ ID NO:12), GRGDY (SEQ ID NO:17), GRGDSP (SEQ ID NO:18), GGGGRGDSP (SEQ ID NO:20) and GGGGRGDY (SEQ ID NO:21). Suitable cell adhesion peptides comprising RGD include, but are not limited, to NOVATACH RGD (NovaMatrix, FMC BioPolymer, Oslo, Norway) and those disclosed in U.S. Pat. No. 6,642,363, which is hereby incorporated by reference in its entirety. Peptide synthesis services are available from numerous companies, including Commonwealth Biotechnologies, Inc. of Richmond, Va., USA. Chemical techniques for coupling peptides to the alginate backbones may be found in U.S. Pat. No. 6,642,363.

As used herein, the term “cell adhesion sequence modified alginate” means an alginate covalently bound to at least one peptide comprising one or more cell adhesion sequences.

As used herein, the term “cell adhesion sequence modified chitosan” means an chitosan covalently bound to at least one peptide comprising one or more cell adhesion sequences.

As used herein, the term “cell adhesion sequence modified hyaluronate” means a hyaluronate covalently bound to at least one peptide comprising one or more cell adhesion sequences.

As used herein, the term “a RGD modified polysaccharide” means a polysaccharide covalently bound to at least one peptide comprising one or more RGD sequences.

As used herein, the term “an RGD modified alginate” refers to an alginate which is covalently linked to a peptide comprising RGD. Suitable RGD peptide coupled alginates include, but are not limited, to NOVATACH RGD (NovaMatrix, FMC BioPolymer, Oslo, Norway) and those disclosed in U.S. Pat. No. 6,642,363, which is hereby incorporated by reference in its entirety.

As used herein, the term “an RGD modified chitosan” refers to a chitosan which is covalently linked to a peptide comprising RGD.

As used herein, the term “an RGD modified hyaluronate” refers to a hyaluronate which is covalently linked to a peptide comprising RGD.

As used herein, the term “weight-average molecular weight” refers to molecular weight determined by Size Exclusion Chromatography with Multiple Angle Laser Light Scatter Detection (SEC-MALS).

As used herein, the term “composite” may refer to a composition wherein each component are the same or different materials.

As used herein, the term “low viscosity solution” in relation to the first liquid component refers to a concentration of polysaccharide of less than about 2% w/v.

As used herein in the context of the gel-forming ions in the foam, the term “gel-forming ions” refers to ions that are capable of forming a gel with the polysaccharide or which do not form a soluble salt with the polysaccharide of the gel. These gel-forming ions are preferably divalent or trivalent ions.

As used herein in the context of the gel-forming ions in the coating, the term “gel-forming ions” refers to ions that are capable of forming a gel with the alginate, modified alginate, or combination thereof, or which do not form a soluble salt with the alginate, modified alginate, or combination thereof. These gel-forming ions are preferably calcium, strontium, or barium ions.

As used herein, the term “completely covers” means that the coating covers the surface such that there are no visible gaps in the coverage, or such that there is no loss in immunogenicity of the composite. That is, the foam is completely coated to the extent that gel within the foam is separated from the immune system of an individual upon implantation of the composite. In some embodiments, the coating is a hydrogel.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

In one aspect, the present invention provides a composite comprising:
i) a foam having pores, wherein said foam comprises gel-forming ions distributed through at least a part of the foam;
ii) a gel comprising a polysaccharide with gelling sites, wherein the gel is located within the pores of the foam and interacts with the foam; and
iii) a coating comprising a polysaccharide with gelling sites and gel-forming ions, wherein said coating completely covers the outer surface of said foam.

Preferably, the gel in the composite described herein will have good structural integrity for its intended use, for example the gel does not leak from the foam unless desired. The composite may be employed to carry functional components, for example pharmaceuticals and cell populations and provides desirable delivery characteristics, for example release of the material to be released. The release may be triggered in any suitable manner, for example by contact with a solvent, by temperature change or by mechanical manipulation. The composite may advantageously be employed to immobilize cells. The components of a composite according to the invention for treatment of the human or animal body are desirably biocompatible and optionally biodegradable.

The gel forms inside the pores of the foam as a result of interaction between an added liquid that contains gel-forming polysaccharide with gelling sites and the gelling ions of the foam. The gelling ions in the pores of the foam in (i) are present to induce gelling of the gel-forming polysaccharide of (ii). The gel forms within the pores and the gel/foam combination component is covered with a coating to form a barrier that protects against immunological attack.

In some embodiments, the elastic modulus of the composite is from 0.1 kPa to 1000 kPa.

In some embodiments, the composite has two sides with each side having an area of about 1 cm². In some embodiments, the composite has two sides with each side having an area from about 0.5 cm² to about 2 cm². In some embodiments, the composite is about 0.5 mm to about 3.0 mm in width. In some embodiments, the composite is about 0.5 mm to about 2.0 mm in width. In some embodiments, the foam is about 1 mm in width. In some embodiments, the foam is from about 0.5 mm to about 2 mm in width. In some embodiments, the foam is from about 0.5 mm to about 3 mm in width.

In some embodiments, the foam comprises a polymer. In some embodiments the foam comprises a biopolymer. In some embodiments the foam comprises a polysaccharide. In some embodiments the foam comprises a polymer selected from the group consisting of: an alginate, a pectin, a carrageenan, a hyaluronate, chitosan, a cell adhesion sequence modified polysaccharide, a RGD modified alginate and combination thereof.

Suitable foams include those having open pore networks preferably having a pore size from 5 to 1000 microns, from 25 to 1000 microns, more preferably in the range of 25 to 500 microns, capable of absorbing an added liquid component containing a polysaccharide into its pores. In some embodiments, the average pore size of the foam is about 50 μm to about 250 μm in diameter. Suitable foams for use in the present invention have pores open on at least one surface and desirably have at least a portion of interconnected pores to enable transport of the absorbed polysaccharide within the foam and/or effectively increase the volume of liquid component which can be absorbed by the foam.

The foam is suitably swellable and preferably may absorb up to 30 times its weight, more preferably from 1 to 20 times its weight of a liquid, for example an aqueous physiological solution or a polysaccharide solution. The foam can have a homogeneous or heterogenous distribution of pore sizes. Not all pores are required to absorb the liquid component.

In some embodiments, the foam absorbs from 1 to 30 times its weight of a liquid selected from the group water, a physiological solution, a soluble polysaccharide solution, and a polysaccharide solution comprising a functional component.

In some embodiments, the composite or foam is sterilized. In some embodiments, the foam is sterilized. In some embodiments, the sterilization comprises γ-irradiation, E-beam, ethylene oxide, autoclaving or contacting the foam with alcohol prior to addition of the liquid component or contacting with NOx gases, hydrogen gas plasma sterilization. Sterilisation should not be employed where it adversely affects the composite, or a functional component contained in the composite.

The foam is preferably dried absorbent foam. A dried absorbent foam having an open pore network and pores and comprising gel-forming ions for gelling a subsequently added polysaccharide solution can be formed by a method comprising:

a) forming a wet foam from an aqueous dispersion or solution comprising a polysaccharide, and a foaming agent and optionally one or more of a plasticizer, a cross-linking agent and a pH modifier;

b) mixing a foam from the aqueous dispersion, optionally by mechanical agitation;

c) optionally carrying out one or more steps of:
i) molding or shaping the foam; and
ii) forming a cross-linked foam from the foam;

d) drying the foam to form a dried foam containing open pores; and

e) adding gel-forming ions in one or more of steps a) to d) or after step d).

In an especially preferred embodiment, the gel-forming ions are added in step a) to provide a substantially uniform distribution of the said gel-forming ions throughout the foam.

Known pH modifiers to reduce or increase pH and plasticizers may be employed for example, those described in WO2005023323.

The foam may be dried by air drying and as desired may be subjected to molding, shaping or compression.

The foam may be prepared using a single foam preferably or alternatively from a heterogenous structure comprising foams of differing density or pore size, and foam and non-foam regions.

The polymer in the foam may be ionically or covalently cross-linkable but does not need to be cross-linkable provided that the polysaccharide of the gel is cross-linkable with the polymer in the foam or with a component of the foam, for example the gel-forming ions.

In some embodiments, the polymer of said foam is a biopolymer. In a preferred embodiment, the foams are formed from biopolymers which are derived from plants or animals. Such foams can be made according to prior art processes, for example, as disclosed in U.S. Pat. No. 5,888,987 (Hayes) or WO2005023323 (Gaserod), U.S. Pat. No. 6,203,845 (Qin) or U.S. Pat. No. 6,656,974 (Renn), each of which is hereby incorporated by reference in its entirety.

In some embodiments, the polymer used for the foam matrix comprises a polysaccharide which is gelled with gel-forming ions. In a preferred embodiment the foam comprises a crosslinked biopolymer optionally containing a foaming agent for example as described in WO2005023323.

The foam preferably comprises a polysaccharide and/or a modified polysaccharide.

In some embodiments, the polymer in the foam is selected from the group consisting of an alginate, a pectin, a carrageenan, a hyaluronate, a chitosan, and combination thereof. In some embodiments, the polymer is selected from the group consisting of an alginate, chitosan, and a hyaluronate. In some embodiments, the polymer comprises an alginate or modified alginate. In some embodiments, the polymer comprises an alginate.

In some embodiments, the polymer is a cell adhesion sequence modified polysaccharide. In some embodiments, the polymer is selected from the group consisting of a cell adhesion sequence modified alginate, a cell adhesion sequence modified chitosan, a cell adhesion sequence modified hyaluronate, and combination thereof. In some embodiments, the polymer comprises a cell adhesion sequence modified polysaccharide. In some embodiments, the polymer comprises a cell adhesion sequence modified alginate. In some embodiments, the polymer comprises a cell adhesion sequence modified chitosan. In some embodiments, the polymer comprises a cell adhesion sequence modified hyaluronate.

In some embodiments, the polymer is a RGD modified polysaccharide, In some embodiments. In some embodiments, the polymer is selected from the group consisting of a RGD modified polysaccharide, a RGD modified alginate, a RGD modified chitosan, a cell adhesion sequence modified hyaluronate, and combination thereof. In some embodiments, the polymer comprises a RGD modified polysaccharide. In some embodiments, the polymer comprises a RGD modified alginate. In some embodiments, the polymer comprises a RGD modified chitosan. In some embodiments, the polymer comprises a RGD modified hyaluronate.

In some embodiments, the polymer has an average molecular weight of about 4 kDa to about 300 kDa. In some embodiments, the polymer has an average molecular weight of about 10 kDa to about 500 kDa. In some embodiments, the polymer has an average molecular weight of about 50 kDa to about 300 kDa.

In some embodiments, the polymer comprises an alginate with an average molecular weight of about 4 kDa to about 300 kDa. In some embodiments, the polymer comprises an alginate with an average molecular weight of about 10 kDa to about 500 kDa. In some embodiments, the polymer comprises an alginate with an average molecular weight of about 50 kDa to about 300 kDa.

In some embodiments, the polymer comprises an alginate with a guluronate (G) content of greater than 20%.

In some embodiments, the foam comprises an alginate, the composite further comprising a functional component selected from a pharmaceutical active and/or an anti-adhesive agent.

In some embodiments, the foam further comprises a structural support, particularly a permeable structural support such as a mesh in contact with the foam. Suitable mesh materials include, but are not limited, polyester mesh. The foam can be incorporated in several ways, in a monolayer or bilayer foam configuration. In some embodiments, the mesh can be placed on the surface of the foam in a monolayer configuration, before forming the gel. The gel will help hold the mesh in place. The structure during addition of the coating layer. In some embodiments, a wet foam is placed on top of a mesh placed in a mold, and then dried. In some embodiments, the mesh is placed on top of a wet foam before drying. In some embodiments, a layer of wet foam is molded, the mesh is added on top of the wet foam, and is then covered by another layer of wet foam. When a bilayer configuration is used, the two foam compositions may be the same or different. The two foam compositions of the bilayer configuration, whether the same or different, may have the same pore size or different pore size from each other. Alternatively, the structural support may be indirectly in contact with the foam such as, for example, by adherence to the coating over the foam or by adherence to another material adhered to the foam.

In some embodiments, the mesh has a mesh opening of about 10 μm to about 1 mm. In some embodiments, the mesh has a mesh opening of about 20 μm to about 500 μm. In some embodiments, the mesh has a mesh opening of about 300 μm.

The inclusion of the mesh may increase the structural integrity of the foam matrix, and may make it easier to implant and remove post implantation.

The foam having pores may be made by any known method in the art, including, but not limited to, mechanical agitation, freeze-drying, knitting, weaving, or layering of fibers. Preferably, the foam is suitably prepared using a mixer, for example a kitchen aid mixer equipped with a wire whisk to aerate an aqueous solution of the polymer for producing the foam together with other components such as plasticizers for example, glycerin and sorbitol.

A foaming agent may be included in the aqueous dispersion to aid in foaming. When present, the foaming agent suitably produces a wet foam resistant to foam collapse. The foaming agent may be a single material or a mixture of materials that aid in foaming. The foaming agent may be a polymeric foaming agent, a surfactant, or a mixture thereof.

Polymeric foaming agents, such as surface active hydrocolloids, are generally preferred for most biological applications because they are harder to leach from the resulting gelled foam than surfactants. Examples of surface active hydrocolloids include methyl cellulose, hydroxy propyl methyl cellulose (HPMC), hydroxy propyl cellulose (HPC), hydroxy ethyl cellulose (HEC), albumin and glycol alginates, such as propylene glycol alginate. For some applications, it may be advantageous to add an additional polysaccharide, for example a cellulose derivative such as carboxymethyl cellulose, in addition to the foaming agent. The polymeric foaming agent is preferably soluble in water so that a homogeneous gelled foam is produced. Preferred water soluble foaming agents include albumin and hydroxy propyl methyl cellulose as they produce small bubbles that result in fine pores in the foam.

When dried cross-linked foams containing high levels of calcium are soaked in water, the foam structure typically does not break down due to the high level of crosslinking of the foam. However, the soluble components in the foam, including water soluble foaming agents such as hydroxy propyl methyl cellulose, may diffuse out of the foam. This loss of foaming agent may be prevented in, for example a wound healing application, by use a foaming agent that is not soluble under conditions of use. Some foaming agents form gels at body temperature, for example methyl cellulose forms gels above 35° C. When using a foam that comprises methyl cellulose as the foaming agent in an application in which the foam is at body temperature, the methyl cellulose will stay in the gelled state and remain in the foam and contribute to the wet strength of the foam.

When a polymeric foaming agent such as hydroxypropyl methyl cellulose is used, the concentration of the polymeric foaming agent in the aqueous dispersion is typically about 0.5 wt % to about 6 wt %, preferably about 1 wt % to about 4 wt %, or more preferably about 1.5% to about 2 wt %. This produces a foam that comprises about 3 wt % to about 37 wt %, preferably about 6 wt % to about 25 wt %, more preferably about 6% to about 12.5 wt %, of the polymeric foaming agent, exclusive of water and any additive or additives that may be present in the foam.

For certain applications, a surfactant, with or without an added polymeric foaming agent, may be used as the foaming agent. Surfactants are well known to those skilled in the art and are described, for example, in McCutcheon's Detergents and Emulsifiers, and Laughlin, U.S. Pat. No. 3,929,678, each of which is hereby incorporated herein by reference in its entirety. Nonionic surfactants are typically condensation products of a hydrophobic organic aliphatic or alkyl aromatic compound and hydrophilic ethylene oxide and/or propylene oxide. The length of the resulting polyether chain can be adjusted to achieve the desired balance between the hydrophobic and hydrophilic properties. Nonionic surfactants include, for example, ethoxylates of alkyl phenols containing from about 8 to 18 carbon atoms in a straight- or branched-chain alkyl group, such as t-octyl phenol and t-nonyl phenol with about 5 to 30 moles of ethylene oxide, for example nonyl phenol condensed with about 9.5 moles of ethylene oxide, dinonyl phenol condensed with about 12 moles of ethylene oxide; ethoxylated and propoxylated alcohols, especially C₁₀₋₂₀ alcohols, with 2 to 100 moles of ethylene oxide and/or propylene oxide per mole of alcohol, especially ethoxylates of primary alcohols containing about 8 to 18 carbon atoms in a straight or branched chain configuration with about 5 to 30 moles of ethylene oxide, for example, the ethoxylates of decyl alcohol, cetyl alcohol, lauryl alcohol, or myristyl alcohol; ethoxylates of secondary aliphatic alcohols containing 8 to 18 carbon atoms in a straight or branched chain configuration with 5 to 30 moles of ethylene oxide; condensation of aliphatic alcohols containing about 8 to abut 20 carbon atoms with ethylene oxide and propylene oxide; polyethylene glycol and polyethylene oxide; ethoxylated castor oil (CREMOPHOR® CO 40); ethoxylated hydrogenated castor oil; ethoxylated coconut oil; ethoxylated lanolin; ethoxylated tall oil; ethoxylated tallow alcohol; and ethoxylates of sorbitan esters such as polyoxyethylene sorbitan monolaurate (TWEEN® 20), polyoxyethylene sorbitan monopalmitate (TWEEN® 40), polyoxyethylene sorbitan monostearate (TWEEN® 60), polyoxyethylene sorbitan monooleate (TWEEN® 80), and polyoxyethylene sorbitan trioleate (TWEEN® 85). For physical applications such as wound dressings, when a surfactant is included in the dried gelled foam, non-ionic surfactants, such as the ethoxylates of sorbitan esters, are preferred. Examples of anionic surfactants are sodium stearate, sodium cetyl sulfate, sodium lauryl sulfate, ammonium lauryl sulfate, triethanolamine lauryl sulfate, sodium myristyl sulfate, and sodium stearyl sulfate, triethanol amine dodecylbenzenesulfonate, sodium dodecylbenzene sulfonate, sodium polyoxyethylene lauryl ether sulfate, and ammonium polyoxyethylene lauryl ether sulfate. A preferred anionic surfactant is sodium lauryl sulfate (sodium dodecyl sulfate). Cationic surfactants include, for example, quaternary ammonium salts, such as cetyl trimethylammonium bromide, lauryl trimethyl ammonium chloride, alkyl benzyl methyl ammonium chlorides, alkyl benzyl dimethyl ammonium bromides, cetyl pyridinium bromide, and halide salts of quaternized polyoxyethylalkylamines. Zwitterionic surfactants can also be used.

When the surfactant is used with a polymeric foaming agent, a useful surfactant is a sorbitan ester, such as TWEEN® 20 surfactant. When a surfactant, such as TWEEN® 20 surfactant, is used with a polymeric foaming agent, the dried gelled foam may comprise about 0.05 wt % to 1.0 wt %, typically 0.1 wt % to 0.5 wt %, of the surfactant. However, for certain applications, such as oral care applications in which a surfactant, such as, for example, sodium lauryl sulfate, is used without a polymeric foaming agent, the dried gelled foam may comprise about 0.5 wt % to 5.0 wt %, typically 1.5 wt % to 3.0 wt %, of the surfactant, excusive of water and any additive or additives, such as silica or other abrasives or polishing agents, that may be present in the foam.

Suitable polysaccharides for use in forming the gel include those that are soluble in a solvent, such as water, and can be formed into a gel by interaction with gel-forming ions. Examples of suitable polysaccharides include alginates, pectins, carrageenans, chitosan, hyaluronates, and mixtures thereof provided that the polysaccharide alone or in a mixture with another polysaccharide may form a gel. Alginates and modified alginates are a preferred polysaccharide for use in the present invention. Modified polysaccharides, also known as polysaccharide derivatives, may be employed in applications of the present invention so long as they are reactive with gel-forming ions.

In some embodiments, the polysaccharide in said gel is selected from the group consisting of an alginate, a chitosan, a carrageenan, a hyaluronate, a pectin, and combination thereof. In some embodiments, the polysaccharide in said gel is selected from the group consisting of an alginate, chitosan, and hyaluronate.

In some embodiments, the polysaccharide in said gel is a cell adhesion sequence modified polysaccharide. In some embodiments, the polysaccharide in said gel is selected from the group consisting of a cell adhesion sequence modified alginate, a cell adhesion sequence modified chitosan, a cell adhesion sequence modified hyaluronate, and combination thereof. In some embodiments, the polysaccharide in said gel comprises a cell adhesion sequence modified polysaccharide. In some embodiments, the polysaccharide in said gel comprises a cell adhesion sequence modified alginate. In some embodiments, the polysaccharide in said gel comprises a cell adhesion sequence modified chitosan. In some embodiments, the polysaccharide in said gel comprises a cell adhesion sequence modified hyaluronate.

In some embodiments, the polysaccharide in said gel is a RGD modified polysaccharide. In some embodiments, the polysaccharide in said gel is selected from the group consisting of a RGD modified alginate, a RGD modified chitosan, a cell adhesion sequence modified hyaluronate, and combination thereof. In some embodiments, the polysaccharide in said gel comprises a RGD modified polysaccharide. In some embodiments, the polysaccharide in said gel comprises a RGD modified alginate. In some embodiments, the polysaccharide in said gel comprises a RGD modified chitosan. In some embodiments, the polysaccharide in said gel comprises a RGD modified hyaluronate.

In some embodiments, the foam and/or the polysaccharide in said gel or said coating comprises an ultrapure polysaccharide possessing a low content of endotoxins. In some embodiments, the foam and/or polysaccharide in said gel or said coating comprises an alginate with an endotoxin content of less than 100 EU/g.

In some embodiments, the polysaccharide in said gel comprises an alginate.

In some embodiments, the polysaccharide in said gel comprises an alginate with a G content of about 20% to about 80%. In some embodiments, the polysaccharide in said gel comprises an alginate with a M content of at least 20%.

In some embodiments, the polysaccharide in said gel comprises an alginate with an average molecular weight from about 4 kD to about 1000 kD, about 4 kD to about 500 kD, or about 4 kD to about 300 kD.

In some embodiments, the polysaccharide in said gel comprises an alginate with a viscosity of about 10 mPas to about 200 mPas In some embodiments, the polysaccharide in said gel comprises an alginate with a viscosity of about 2 mPas to about 500 mPas In some embodiments, the polysaccharide in said gel is selected from an alginate, a carrageenan, a hyaluronate, a pectin, and combination thereof; and the gel-forming ions in said foam are selected from the group consisting of calcium ions, barium ions, strontium ions, or combination thereof.

In some embodiments, the polysaccharide in said gel comprises a chitosan; and the gel-forming ions in said foam are selected from the group consisting of triphosphate ions, phosphate ions, citrate ions, or combination thereof.

The coating of the composite is present such the outer surface of the foam, including any gel on the surface of the foam, is completely covered by the coating. While not wishing to be bound by any particular theory, it is believed that the coating is immunoprotective and reduces the likelihood of fibrosis.

In some embodiments, the polysaccharide of the coating comprises chitosan, hyaluronate, or alginate.

In some embodiments, the polysaccharide in said coating is a cell adhesion sequence modified polysaccharide. In some embodiments, the polysaccharide in said coating is selected from the group consisting of a cell adhesion sequence modified alginate, a cell adhesion sequence modified chitosan, a cell adhesion sequence modified hyaluronate, and combination thereof. In some embodiments, the polysaccharide in said coating comprises a cell adhesion sequence modified polysaccharide. In some embodiments, the polysaccharide in said coating comprises a cell adhesion sequence modified alginate. In some embodiments, the polysaccharide in said coating comprises a cell adhesion sequence modified chitosan. In some embodiments, the polysaccharide in said coating comprises a cell adhesion sequence modified hyaluronate.

In some embodiments, the polysaccharide in said coating is a RGD modified polysaccharide. In some embodiments, the polysaccharide in said coating is selected from the group consisting of a RGD modified alginate, a RGD modified chitosan, a cell adhesion sequence modified hyaluronate, and combination thereof. In some embodiments, the polysaccharide in said coating comprises a RGD modified polysaccharide. In some embodiments, the polysaccharide in said coating comprises a RGD modified alginate. In some embodiments, the polysaccharide in said coating comprises a RGD modified chitosan. In some embodiments, the polysaccharide in said coating comprises a RGD modified hyaluronate.

In some embodiments, the coating comprises an alginate with a weight-average molecular weight of about 4 kD to about 300 kD. In some embodiments, the coating comprises an alginate with a weight-average molecular weight of about 50 kD to about 300 kD. In some embodiments, the coating comprises an alginate with a weight-average molecular weight of about 150 kD to about 250 kD. In some embodiments, the coating comprises an alginate with a weight-average molecular weight of about 75 kD to about 150 kD. In some embodiments, the polymer has a weight-average molecular weight of about 50 kD to about 300 kD, about 150 kD to about 250 kD, or about 75 kD to about 150 kD.

In some embodiments, the coating comprises an alginate with a mannuaronate to guluronate ratio equal to or greater than about 1.

In some embodiments, the coating comprises an alginate derived from macrocystitis purifera.

In some embodiments, the coating comprises an alginate selected from the group consisting of PRONOVA SLM₁₀₀ or SLM₂₀ (NovaMatrix, FMC Corp., Oslo, Norway).

In some embodiments, the coating comprises a modified alginate, wherein said modified alginate comprises at least one alginate chain section to which is bonded by covalent bonding at least one cell attachment peptide containing one or more RGD sequences.

A salt or combination of salts that provides the desired gel-forming ions or mixture of gel-forming ions may be used as the gel-forming ions. Suitable gel-forming ions for forming the gel or the coating include monovalent and polyvalent ions, preferably a divalent and/or a trivalent ions, or mixture of ions capable of forming a gel with the polysaccharide or which do not form a soluble salt with the polysaccharide. Gel-forming ions for specific polysaccharides are known from the literature. For alginates, suitable polyvalent cations include, for example, calcium(2+), barium(2+), strontium(2+), iron(2+), zinc(2+), copper(2+), and aluminum(3+). Preferred cations are divalent metal cations, more preferably the calcium (2+) cation. A monovalent cation such as potassium would not be considered a gelling ion for an alginate since potassium alginate is a soluble alginate salt; however, the potassium cation would be a suitable gelling ion for kappa carrageenan or kappa II carrageenan. Where the polysaccharide salt is positively charged, for example, chitosan, negatively charged gel-forming ions, for example phosphates or citrates may be employed. In some embodiments, the gel-forming ions comprise a phosphate or a citrate ion, wherein the polysaccharide in the gel is chitosan.

In some embodiments, the polysaccharide in said coating is selected from an alginate, a hyaluronate, and combination thereof; and the gel-forming ions in said foam are selected from the group consisting of calcium ions, barium ions, strontium ions, or combination thereof. In some embodiments, the polysaccharide in said coating is selected from an alginate, a hyaluronate, and combination thereof, and the gel-forming ions in said coating are each independently selected from the group consisting of strontium ions, barium ions, calcium ions, and combination thereof. In some embodiments, the polysaccharide in said coating is selected from an alginate, a hyaluronate, and combination thereof; and the gel-forming ions in said coating are each independently selected from the group consisting of strontium ions, calcium ions, and combination thereof.

In some embodiments, the polysaccharide in said coating is an alginate; and the gel-forming ions in said foam are selected from the group consisting of calcium ions, barium ions, strontium ions, or combination thereof. In some embodiments, the polysaccharide in said coating is an alginate; and the gel-forming ions in said coating are each independently selected from the group consisting of strontium ions, barium ions, calcium ions, and combination thereof. In some embodiments, the polysaccharide in said coating is an alginate; and the gel-forming ions in said coating are each independently selected from the group consisting of strontium ions, calcium ions, and combination thereof.

In some embodiments, the polysaccharide in said coating comprises a chitosan; and the gel-forming ions in said foam are selected from the group consisting of triphosphate ions, phosphate ions, citrate ions, or combination thereof.

In some embodiments, the coating comprises about 0.2% w/v to about 10%, about 0.5% w/v to about 10%, 1% w/v to about 8% w/v, about 2% to about 7%, about 3% to about 6%, or about 2% w/v to about 6% w/v of a polysaccharide with gelling sites. In some embodiments, the polysaccharide of said second liquid component comprises about 3% w/v by weight of a polysaccharide with gelling sites. In some embodiments, the coating comprises about 5% w/v by weight of a polysaccharide with gelling sites. In some embodiments, the coating comprises about 5% w/v by weight of PRONOVA SLM₂₀ (NovaMatrix, FMC Biopolymer, Norway. In some embodiments, the coating comprises about 5% w/v by weight of PRONOVA SLM₁₀₀ (NovaMatrix, FMC Biopolymer, Norway).

In some embodiments, the coating comprises about 0.2% w/v to about 10%, about 0.5% w/v to about 10%, 1% w/v to about 8% w/v, about 2% to about 7%, about 3% to about 6%, or about 2% w/v to about 6% w/v of an alginate, cell sequence modified alginate, or combination thereof. In some embodiments, the polysaccharide of said second liquid component comprises about 3% w/v of an alginate, cell sequence modified alginate, or combination thereof. In some embodiments, the coating comprises about 5% w/v of an alginate, cell sequence modified alginate, or combination thereof. In some embodiments, the coating comprises about 5% w/v by weight of PRONOVA SLM₂₀ (NovaMatrix, FMC Biopolymer, Norway. In some embodiments, the coating comprises about 5% w/v by weight of PRONOVA SLM₁₀₀ (NovaMatrix, FMC Biopolymer, Norway).

The gel-forming ions may be able to form a gel with the polymer of the foam and/or the polysaccharide. The gel-forming ions may form links between the foam and the soluble polysaccharide. Preferably, the “gel-forming ions” in the foam are donatable to the polysaccharide and are present in the foam at a level such that at least some of the gelling sites of the polysaccharide are occupied upon contacting the liquid component to the foam. Suitably, the gel-forming ions may be present in the foam at a sub-stoichiometric, stoichiometric or super-stoichiometric level with respect to sites in the foam for binding the gel-forming ions provided that sufficient gel-forming ions are present to occupy at least some of the gelling sites in the polysaccharide to be added.

In one embodiment, the gel-forming ions are not able to form a gel with the polymer of the foam.

In another aspect, the foam may comprise an excess of gel-forming ions relative to the gelling sites in the soluble polysaccharide. At least some of the gel-forming ions may be incorporated into the foam prior to addition of a soluble polysaccharide which then gels by interaction with the gel-forming ions within the foam structure. In some embodiments, the foam does not comprise an excess of gel-forming ions relative to the gelling sites in the soluble polysaccharide.

The concentration of gel-forming ions in the foam or coating may be controlled so that the resulting gel or coating contains polysaccharide or alginate with gelling sites that are not fully reacted with gel-forming ions; i.e., the gel-forming ions or mixture of gel-forming ions is present in a molar amount less than that required to saturate 100% of the gelling sites of the polysaccharide. For example, when sufficient gel-forming ions, such as calcium ion, are present to react with all available gelling sites (eg. the L-guluronic acid units in the case of alginate, D-galacturonic acid units in the case of pectin substances), the gel-forming polysaccharide or alginate is 100% saturated. The amount of cation required to completely saturate the gelling sites of alginate, for example, is considered to be 1 mole of divalent cation per 2 moles of L-guluronic acid in the alginate or 1 mole of trivalent cation per 3 moles of L-guluronic acid in the alginate when only a divalent cation or only a trivalent cation is used in the gelling. When a mixture of a divalent cation or cations and a trivalent cation or cations is used, the amounts required to saturate the alginate can be determined because a divalent cation occupies two gelling sites and a trivalent cation occupies three gelling sites. Thus, any amount less than this is considered to be an amount less than that required to completely saturate the gelling sites of the alginate. Suitably, the gel-forming ions present in the foam are sufficient to saturate about 5% to 250%, more suitably 5% to 200%, preferably about 35% to 150%, even more preferably about 50% to 100%, of the gelling sites of the polysaccharide.

The foam itself may be prepared using a polysaccharide and also requires gel-forming ions. In the case where both the foam and the polysaccharide rely on the same gel-forming ions, the foam alone may have an initial saturation when prepared, for example, of 150%, however when additional polysaccharide is added as a liquid and absorbed into the pores of the foam, some of the gel-forming ions are used to gel the added polysaccharide. In this case, the saturation of the added polysaccharide is calculated based on the total amount of gel-forming ions and the total amount of gelling sites for both the polysaccharide in the foam and the added polysaccharide forming the gels in the pores.

In some embodiments, the gel-forming ions in said foam are present in a molar amount equivalent to at least 10% of said gelling sites of said polysaccharide of said gel. In some embodiments, the gel-forming ions in said foam are present in a molar amount equivalent to 5% to 250% of said gelling sites of said polysaccharide of said gel. In some embodiments, the gel-forming ions in said foam are comprise calcium ions and are present in a molar amount equivalent to 5% to 200% of said gelling sites of said polysaccharide of said gel. In some embodiments, the gel-forming ions in said foam are comprise strontium ions and are present in a molar amount equivalent to at least 5% to 200% of said gelling sites of said polysaccharide of said gel. In some embodiments, the gel-forming ions in said foam are comprise barium ions and are present in a molar amount equivalent to at least 5% to 200% of said gelling sites of said polysaccharide of said gel.

For alginate, the strength of gels formed by reaction of alginate with polyvalent cations is related to the molecular weight of the alginate, the guluronic acid content (“G content”) of the alginate as well as the arrangement of guluronic and mannuronic acids on the polymer chain. In addition, the pore size, and thickness of the foam, the alginate concentration, the level of the gel-forming ions and the type of ions employed also contribute to strength. The G content of the alginate for the gel is suitably at least about 30%, preferably about 40% to about 90%, and more preferably about 50% to about 80%. Alginate derived from, for example, Lessonia trabeculata and from the stems of Laminaria hyperborea have a high G content and may, as a preference, be used to form the gelled foams of the invention. Fully saturated alginates with a high G content give gels with the highest mechanical strength.

The amount of divalent cation, such as calcium, required to react stoichiometrically with these G-blocks can be calculated for each alginate type by considering that two guluronic acid units plus one divalent cation are required to create one ionic crosslink. The amount of calcium required for stoichiometric saturation of a 1% sodium alginate solution are given in the following table:

| Seaweed Source | % G | mM Ca |
|---|---|---|
| Laminaria hyperborea (stem) | 70 | 14-16 |
| Laminaria hyperborea (leaf) | 54 | 11-13 |
| Lessonia trabeculata | 68 | 13-15 |
| Macrocystis pyrifera | 39 | 8-9 |

A list of various commercially available alginates, their properties, and their sources is found in Shapiro, U.S. Pat. No. 6,334,968, Table 1, column 16, line 49, to column 17, line 18, which is hereby incorporated herein by reference in its entirety. Mixtures or blends of alginates, for example alginates of different molecular weights and/or G content, may be used as the gel-forming polymer.

Complete saturation (100% saturation) of the gelling sites occurs when the composition contains 1 mole of divalent cation per 2 moles of L-guluronic acid units. For example, an about 15 mM solution of calcium ion is required to 100% saturate a 1% solution of sodium alginate extracted from the stems of Laminaria hyperborea, an about 12 mM calcium solution is required to 100% saturate a 1% solution of sodium alginate extracted from the leaves (fronds) of Laminaria hyperborea, and an about 14 mM solution of calcium ions is required to 100% saturate a 1% solution of sodium alginate extracted from Lessonia trabeculata. Thus, when alginate is used as the gel-forming or coating forming polymer, the gel-forming composition preferably comprises 0.2 to 0.9 mM of divalent cation, preferably 20% to 90% calcium (2+) ion, per 2 mM of L-guluronic acid units present in the alginate. When using a sparingly soluble salt as the gel-forming ions, the extent of cross-linking can be controlled by controlling either the amount of gelling agent, for example, calcium carbonate, and/or the amount of solubilizing agent, for example a pH modifier such as glucono delta-lactone, present during gel formation. Preferably, there should be a stoichiometric relationship between the pH modifier and the gelling agent such that substantially all gel-forming ions are available.

When all the gelling sites on the polysaccharide or alginate of the gel and coating, respectively, are not saturated with gel-forming ions, the remaining sites are occupied by non-cross-linking ions. If desired, active ions, such as the Ag(1+) cation, may be used to occupy some or all of the remaining sites. Scherr, U.S. 2003/0021832 A1, discloses that silver alginate may be used for the treatment of burns, wounds, ulcerated lesions, and related pathological states.

The gel or first liquid component containing the polysaccharide may further include a functional component which is suitably disposed in, for example entrapped in the polysaccharide gel. Any functional component may be added so long as it does not prevent the first liquid component from being absorbed into the foam or the polysaccharide from forming a gel. The functional component may be a liquid or a solid and, if insoluble, is dispersed as fine particles in the liquid. Desirable components to be disposed in the gel include beneficial agents such as flavors, fragrances, pharmaceutical and veterinary medicaments, enzymes, growth modifiers, and probiotics, living cells, including plant cells, animal cells, and human cells, yeasts, bacteria, and the like. Incorporation of pharmaceuticals, particulates, cells, multicellular aggregates, tissue, and the like may require gentle mixing in the polysaccharide, preferably alginate, solution. The component can include heat sensitive materials such as cells, drugs, flavors, or fragrances that may, if desired, later be released from the gel.

In some embodiments, the gel further comprises a functional component. In some embodiments, the functional component is selected from the group comprising a flavor, a fragrance, a particulate, cells, multicellular aggregates, a therapeutic agent, a tissue-regenerative agent, a growth factor, a nutraceutical, and combination thereof. In some embodiments, the gel is inert to or stabilizes said functional component.

In some embodiments, the gel further comprises cells. A wide variety of cells appropriate for use in accordance with the composites described herein, as will be readily appreciated by one of skill in the art of cell implantation. Appropriate cells (autologous, allogeneic, xenogeneic) include, for example, hepatocytes, all types of stem cells, insulin producing cells including cells derived from stem cells of any origin (e.g., pancreatic islet cells, isolated pancreatic beta cells, insulinoma cells, etc.), endocrine hormone-producing cells (e.g., parathyroid, thyroid, adrenal, etc.) and any genetically engineered cells that secrete therapeutic agents, such as proteins or hormones for treating disease or other conditions, and genetically engineered cells that secrete diagnostic agents. In some embodiments, the gel comprises pancreatic islet cells, hepatic cells, neural cells, vascular endothelial cells, parathyroid cells, thyroid cells, adrenal cells, thymic cells, mesenchymal stem cells, and ovarian cells. In some embodiments, the gel comprises cells selected from the group consisting of pancreatic islet cells, mesenchymal stem cells, parathyroid cells, and thyroid cells. In some embodiments, the gel comprises cells selected from the group consisting of pancreatic islet cells and parathyroid cells. In some embodiments, the gel comprises pancreatic islet cells. In some embodiments, the gel comprises mesenchymal stem cells genetically modified to express growth factor or coagulation factor VIII. In some embodiments, the gel comprises mesenchymal stem cells. In some embodiments, the gel further comprises cells, provided that said cells are not embryonic stem cells. In some embodiments, the first liquid component comprises tissue of any of the cells described herein.

In some embodiments, the gel further comprises pancreatic islet cells. In some embodiments, the gel further comprises pancreatic islet cells derived from a human, adult pig, or neonatal pig. In some embodiments, the gel further comprises about 20,000 to about 40,000 pancreatic islet cells.

In some embodiments, the cells comprise cells from a human or pig. In some embodiments, the cells comprise cells from a human, neonatal pig, or an adult pig. In some embodiments, the cells comprise cells from a human. In some embodiments, the cells comprise cells from a neonatal pig, or an adult pig. In some embodiments, the cells comprise cells from a neonatal pig. In some embodiments, the cells comprise cells from an adult pig. In some embodiments, the pancreatic islet cells comprise cells from a human or pig. In some embodiments, the pancreatic islet cells comprise cells from a human, neonatal pig, or an adult pig. In some embodiments, the pancreatic islet cells comprise cells from a human. In some embodiments, the pancreatic islet cells comprise cells from a neonatal pig, or an adult pig. In some embodiments, the pancreatic islet cells comprise cells from a neonatal pig. In some embodiments, the pancreatic islet cells comprise cells from an adult pig.

Where present, the functional component is selected according to the intended use of the composite.

In some embodiments:
i) said polymer in said foam is selected from the group consisting of an alginate, a cell adhesion sequence modified alginate, and combination thereof;
ii) said gel-forming ions in said foam comprise multivalent cations;
iii) said polysaccharide in said gel is selected from the group consisting of an alginate, a cell adhesion sequence modified alginate, and combination thereof; and
iv) said gel-forming ions in said coating comprise multivalent cations.

In some embodiments:
i) said polymer in said foam is an alginate, a cell adhesion sequence modified alginate, and combination thereof;
ii) said gel-forming ions in said foam are selected from the group consisting of calcium ions, strontium ions, barium ions, and combination thereof;
iii) said polysaccharide in said gel is selected from the group consisting of an alginate, a cell adhesion sequence modified alginate, and combination thereof; and
iv) said gel-forming ions in said coating are selected from the group consisting of calcium ions, strontium ions, barium ions, and combination thereof.

In some embodiments:
i) said polymer in said foam is an alginate;
ii) said gel-forming ions in said foam are selected from the group consisting of calcium ions, strontium ions, barium ions, and combination thereof;
iii) said polysaccharide in said gel is an alginate;
iv) said polysaccharide in said coating comprises an alginate; and
v) said gel-forming ions in said coating are selected from the group consisting of calcium ions, strontium ions, barium ions, and combination thereof.

In some embodiments:
i) said polymer in said foam is an alginate;
ii) said gel-forming ions in said foam are selected from the group consisting of calcium ions, strontium ions, barium ions, and combination thereof;
iii) said polysaccharide in said gel is a RGD modified alginate;
iv) said polysaccharide in said coating comprises an alginate; and
v) said gel-forming ions in said coating are selected from the group consisting of calcium ions, strontium ions, barium ions, and combination thereof.

In some embodiments:
i) said polymer in said foam is an alginate;
ii) said gel-forming ions in said foam are selected from the group consisting of calcium ions, strontium ions, and combination thereof;
iii) said polysaccharide in said gel is a RGD modified alginate;
iv) said polysaccharide in said coating comprises an alginate; and
v) said gel-forming ions in said coating are selected from the group consisting of calcium ions, strontium ions, and combination thereof.

In some embodiments:
i) said polymer in said foam is an alginate;
ii) said gel-forming ions in said foam are calcium ions;
iii) said polysaccharide in said gel is a RGD modified alginate;
iv) said polysaccharide in said coating comprises an alginate; and
v) said gel-forming ions in said coating are calcium ions.

In some embodiments:
i) said polymer in said foam is selected from the group consisting of an alginate, a cell adhesion sequence modified alginate, and combination thereof;
ii) said gel-forming ions in said foam comprise multivalent cations;
iii) said polysaccharide in said gel is selected from the group consisting of an alginate, a cell adhesion sequence modified alginate, and combination thereof;
iv) said gel-forming ions in said coating comprise multivalent cations; and
v) the gel further comprises cells.

In some embodiments:
i) said polymer in said foam is an alginate, a cell adhesion sequence modified alginate, and combination thereof;
ii) said gel-forming ions in said foam are selected from the group consisting of calcium ions, strontium ions, barium ions, and combination thereof;
iii) said polysaccharide in said gel is selected from the group consisting of an alginate, a cell adhesion sequence modified alginate, and combination thereof; and
iv) said gel-forming ions in said coating are selected from the group consisting of calcium ions, strontium ions, barium ions, and combination thereof; and
v) the gel further comprises cells.

In some embodiments:
i) said polymer in said foam is an alginate;
ii) said gel-forming ions in said foam are selected from the group consisting of calcium ions, strontium ions, barium ions, and combination thereof;
iii) said polysaccharide in said gel is an alginate;
iv) said polysaccharide in said coating comprises an alginate;
v) said gel-forming ions in said coating are selected from the group consisting of calcium ions, strontium ions, barium ions, and combination thereof; and
vi) the gel further comprises cells.

In some embodiments:
i) said polymer in said foam is an alginate;
ii) said gel-forming ions in said foam are selected from the group consisting of calcium ions, strontium ions, barium ions, and combination thereof;
iii) said polysaccharide in said gel is a RGD modified alginate;
iv) said polysaccharide in said coating comprises an alginate;
v) said gel-forming ions in said coating are selected from the group consisting of calcium ions, strontium ions, barium ions, and combination thereof;
vi) the gel further comprises cells.

In some embodiments:
i) said polymer in said foam is an alginate;
ii) said gel-forming ions in said foam are selected from the group consisting of calcium ions, strontium ions, and combination thereof;
iii) said polysaccharide in said gel is a RGD modified alginate;
iv) said polysaccharide in said coating comprises an alginate;
v) said gel-forming ions in said coating are selected from the group consisting of calcium ions, strontium ions, and combination thereof; and
vi) the gel further comprises cells.

In some embodiments:
i) said polymer in said foam is an alginate;
ii) said gel-forming ions in said foam are calcium ions;
iii) said polysaccharide in said gel is a RGD modified alginate;
iv) said polysaccharide in said coating comprises an alginate;
v) said gel-forming ions in said coating are calcium ions;
vi) the gel further comprises cells.

In some embodiments:
i) said polymer in said foam is an alginate;
ii) said gel-forming ions in said foam are selected from the group consisting of calcium ions, strontium ions, and combination thereof;
iii) said polysaccharide in said gel is an alginate;
iv) said polysaccharide in said coating comprises an alginate;
v) said gel-forming ions in said coating are selected from the group consisting of calcium ions, strontium ions, and combination thereof; and
vi) said composite further comprises a mesh.

In some embodiments:
vii) said polymer in said foam is an alginate;
viii) said gel-forming ions in said foam are selected from the group consisting of calcium ions, strontium ions, and combination thereof,
ix) said polysaccharide in said gel is a RGD modified alginate;
x) said polysaccharide in said coating comprises an alginate;
xi) said gel-forming ions in said coating are selected from the group consisting of calcium ions, strontium ions, and combination thereof; and
xii) said composite further comprises a mesh.

In some embodiments:
i) said polymer in said foam is an alginate;
ii) said gel-forming ions in said foam are selected from the group consisting of calcium ions, strontium ions, and combination thereof;
iii) said polysaccharide in said gel is an alginate;
iv) said polysaccharide in said coating comprises an alginate;
v) said gel-forming ions in said coating are selected from the group consisting of calcium ions, strontium ions, and combination thereof; and
vi) the gel further comprises pancreatic islet cells.

In some embodiments:
i) said polymer in said foam is an alginate;
ii) said gel-forming ions in said foam are selected from the group consisting of calcium ions, strontium ions, and combination thereof;
iii) said polysaccharide in said gel is a RGD modified alginate;
iv) said polysaccharide in said coating comprises an alginate;
v) said gel-forming ions in said coating are selected from the group consisting of calcium ions, strontium ions, and combination thereof; and
vi) the gel further comprises pancreatic islet cells.

In some embodiments:
i) said polymer in said foam is an alginate;
ii) said gel-forming ions in said foam are selected from the group consisting of calcium ions, strontium ions, and combination thereof;
iii) said polysaccharide in said gel is an alginate;
iv) said polysaccharide in said coating comprises an alginate; wherein said alginate comprises about 0.5% to about 7% of said coating;
v) said gel-forming ions in said coating are selected from the group consisting of calcium ions, strontium ions, and combination thereof; and
vi) the gel further comprises cells.

In some embodiments:
i) said polymer in said foam is an alginate;
ii) said gel-forming ions in said foam are selected from the group consisting of calcium ions, strontium ions, and combination thereof;
iii) said polysaccharide in said gel is a RGD modified alginate;
iv) said polysaccharide in said coating comprises an alginate; wherein said alginate comprises about 0.5% to about 7% of said coating;
v) said gel-forming ions in said coating are selected from the group consisting of calcium ions, strontium ions, and combination thereof; and
vi) the gel further comprises cells.

In some embodiments:
i) said polymer in said foam is an alginate;
ii) said gel-forming ions in said foam are selected from the group consisting of calcium ions, strontium ions, and combination thereof;
iii) said polysaccharide in said gel is an alginate;
iv) said polysaccharide in said coating comprises an alginate; wherein said alginate comprises about 0.5% to about 7% of said coating;
v) said gel-forming ions in said coating are selected from the group consisting of calcium ions, strontium ions, and combination thereof; and
vi) the gel further comprises pancreatic islet cells.

In some embodiments:
i) said polymer in said foam is an alginate;
ii) said gel-forming ions in said foam are selected from the group consisting of calcium ions, strontium ions, and combination thereof;
iii) said polysaccharide in said gel is a RGD modified alginate;
iv) said polysaccharide in said coating comprises an alginate; wherein said alginate comprises about 0.5% to about 7% of said coating;
v) said gel-forming ions in said coating are selected from the group consisting of calcium ions, strontium ions, and combination thereof; and
vi) the gel further comprises pancreatic islet cells.

In some embodiments, the composite has been equilibrated in a solution of about 1.8 mM calcium. In some embodiments, the composite has been equilibrated in a solution comprising a physiological concentration of calcium ions.

In some embodiments, the foam comprises an alginate, the composite further comprising a functional component selected from a pharmaceutical active and/or an anti-adhesive agent.

In some embodiments, the foam absorbs from 1 to 30 times its weight of a liquid selected from the group water, a physiological solution, a soluble polysaccharide solution, and a polysaccharide solution comprising a functional component.

### Methods of Producing Composites

The present invention further provides a method of producing any of the composites of the invention, including the various composite embodiments described herein and combinations of those embodiments, comprising:
iii) contacting a first liquid component with a foam having pores, wherein said first liquid component comprises a polysaccharide having gelling sites capable of forming a gel upon contact with said ions, whereby said gel forms upon contact with the said gel-forming ions; and
iv) forming said coating on the outer surface of said foam;
   wherein:

said foam comprises gel-forming ions, wherein said ions are distributed through at least a part of the foam; and

said coating comprises a polysaccharide with gelling sites and gel-forming ions, wherein said coating completely covers the outer surface of said foam.

In some embodiments, the forming of the coating comprises:
i) applying a second liquid component comprises a polysaccharide with gelling sites to the outer surface of the foam; and
ii) after said applying, contacting said second liquid component with a solution of gel-forming ions to form said coating.

The methods described herein can be used to the produced any of the embodiments of the composites described herein, or combinations of those embodiments.

In some embodiments, the foam further comprises a polymer.

The method suitably includes absorbing a first liquid component containing a polysaccharide into a foam having pores where gel-forming ions are incorporated in the foam, and gelling the polysaccharide within pores of the foam. The polysaccharide in the first liquid component is suitably reactive with the gel-forming ions in the foam so as to form the gel.

The foam comprises the gel-forming ions and upon addition of the first liquid containing the polysaccharide, the polysaccharide may advantageously penetrate to the interior of the foam via pores and channels in the foam and react with the gel-forming ions so as to form the composite. In this way the gel is formed through at least a part of the interior volume of the foam. Addition of the polysaccharide to the foam before incorporating the gel-forming ions is disadvantageous in that on adding the said ions, reaction is likely to take place on or near the surface of the foam so forming a gel layer which may impede penetration of said ions to the interior volume of the foam. Furthermore, the gel and the composite may be undesirably inhomogeneous. In a preferred embodiment, the composite comprises a substantially homogeneous gel.

Gel-forming ions for reacting with the first liquid component comprising the polysaccharide may be incorporated during preparation of the foam or added to the foam preferably prior to addition of the liquid component. The gel-forming ions may be incorporated by being dispersed within a mixture, preferably a biopolymer mixture, prior to formation of the mixture into a wet foam or added to the formed foam. Optionally, additional gel-forming ions may be added to the composite comprising a foam and the gel comprising the added polysaccharide. Gel-forming ions may be incorporated into the foam or mixture for making the foam for example, by washing or soaking the foam with a gelling ion solution which does not dissolve the foam. Excess solution may be removed by compressing.

By providing gel-forming ions in the foam prior to introduction of the liquid component comprising a polysaccharide, the polysaccharide may interact with the ions such that on addition of the liquid component comprising the polysaccharide, a gel forms in at least part of and preferably through substantially all of the internal volume of the foam. By providing sufficient gel-forming ions in the foam prior to the addition of the liquid component comprising the polysaccharide a composite having a gel formed through at least a part of the volume of the composite may be secured.

Typical washing solutions for the polysaccharide foam have about 30 mM to about 200 mM, more preferably from 50 to 100 mM, of a water-soluble gelling salt such as calcium chloride, barium chloride, or strontium chloride. Suitably, the rate of gelation may be controlled to delay gelling by using sparingly soluble salts under pH conditions which they are slowly solubilized, or by using soluble gel-forming ions in combination with sequestrants. Washing or soaking can be used to modify the properties of the composite where additional gel-forming ions may be added to strengthen or harden the composite and also to control cell proliferation, while other treatments such as sequestrants or non-gel-forming ions may be used to weaken or dissolve the composite. Alginate gels can be dissolved by addition of an aqueous solution of citrate, EDTA or hexametaphosphate. Wash treatments for use with living cells must be isotonic. The properties of the composite may accordingly be tailored as desired.

Gel formation will depend upon amount and type of gel-forming ions, polysaccharide characteristics such as composition and molecular weight; and liquid component properties relative to absorption such as solids concentration, and viscosity, and the relative proportion of liquid to the available pore volume. In some embodiments, the liquid will be absorbed, the polysaccharide begins to gel, and the gel will fill the pores. Other embodiments, for example, a substantial portion of the polysaccharide may gel as a coating on the pore walls as the liquid is absorbed.

The absorption rate of the first liquid component and the rate of gelling of the polysaccharide may impact the structure of the composite. Absorption rate and absorptive capacity of the liquid component will depend upon foam characteristics such as pore size and volume, polysaccharide characteristics such as composition, and molecular weight; and liquid component properties relative to absorption such as solids concentration, and viscosity. When the viscosity of the first liquid component impacts its ability to be rapidly absorbed into the foam, it may be suitable to use a lower concentration of the alginate or to use an alginate of a lower molecular weight.

Factors which affect the mechanical strength of the gel include the concentration of the polysaccharide, molecular weight of the polysaccharide, the chemical composition, the blend of different polysaccharide components as appropriate, for example the ratio of M-rich and G-rich alginates, the type and level of gel-forming ions and other components of the gel. The strength of the gel may be tailored by manipulating these parameters according to the intended application.

The first liquid component suitable to add to the foam contains the polysaccharide dissolved in a solvent, typically water.

Suitably the viscosity of the first liquid component is from about 5 mPas to about 1000 mPas, more typically about 8 mPas to 600 mPas, more preferably about 10 mPas to 200 mPas. The liquid component is preferably fully absorbed into the pores of the foam unless it is desired to create a gel layer on only a part of the foam, for example on the surface of the foam. The liquid should be sufficiently gelled so it is retained in the pores to avoid un-gelled or partially gelled material from leaking out of the foam unless it is desired to only coat the pores.

In some embodiments, said first liquid component is a low viscosity solution.

In some embodiments, the first liquid component comprises less than about 5% w/v of a polysaccharide. In some embodiments, the first liquid component comprises from about 0.2% w/v to about 10% w/v of a polysaccharide. In some embodiments, the first liquid component comprises from about 0.5% w/v to about 5% w/v of a polysaccharide. In some embodiments, said first liquid component comprises from about 0.1% w/v to about 1.0% w/v of a polysaccharide.

In some embodiments, the foam is washed after said forming of said gel and before said forming of said coating.

In some embodiments, the second liquid component comprises about 0.2% w/v to about 10%, about 0.5% w/v to about 10%, about 1% w/v to about 8% w/v, about 2% to about 7%, about 3% to about 6%, or about 2% w/v to about 6% w/v of a polysaccharide with gelling sites. In some embodiments, the polysaccharide of said second liquid component comprises about 3% w/v by weight of a polysaccharide with gelling sites. In some embodiments, the second liquid component comprises about 5% w/v by weight of a polysaccharide with gelling sites. In some embodiments, the second liquid component comprises about 5% w/v by weight of PRONOVA SLM₂₀ (NovaMatrix, FMC Biopolymer, Norway. In some embodiments, the second liquid component comprises about 5% w/v by weight of PRONOVA SLM₁₀₀ (NovaMatrix, FMC Biopolymer, Norway).

In some embodiments, the second liquid component comprises about 0.2% w/v to about 10%, about 0.5% w/v to about 10%, about 1% w/v to about 8% w/v, about 2% to about 7%, about 3% to about 6%, or about 2% w/v to about 6% w/v of an alginate, cell sequence modified alginate, or combination thereof. In some embodiments, the polysaccharide of said second liquid component comprises about 3% w/v of an alginate, cell sequence modified alginate, or combination thereof. In some embodiments, the second liquid component comprises about 5% w/v of an alginate, cell sequence modified alginate, or combination thereof. In some embodiments, the second liquid component comprises about 5% w/v by weight of PRONOVA SLM₂₀ (NovaMatrix, FMC Biopolymer, Norway. In some embodiments, the second liquid component comprises about 5% w/v by weight of PRONOVA SLM₁₀₀ (NovaMatrix, FMC Biopolymer, Norway).

In some embodiments, the solution of gel-forming ions comprises about 50 to about 200 mM calcium ions. In some embodiments, the solution of gel-forming ions comprises about 100 mM calcium ions.

In some embodiments, the methods further comprises contacting the composite with a solution of strontium ions and calcium ions after said forming of said coating. In some embodiments, the concentration of strontium ions and calcium ions are each independently about 2 to about 8 mM, or about 5 mM. In some embodiments, this step increases the mechanical strength of the resultant composite.

In some embodiments, the methods further comprise washing the composite is equilibrated in a solution comprising about 1.8 mM calcium ions after said forming of said coating. In some embodiments, the methods further comprise washing the composite is equilibrated in a solution comprising about a physiological concentration of calcium ions after said forming of said coating. In some embodiments, this step increases the biocompatibility of the resultant composite, as the concentration of calcium ions is a physiological concentration.

In some embodiments, the first liquid component further comprises a functional component. In some embodiments, the functional component is selected from the group comprising a flavor, a fragrance, a particulate, cells, multicellular aggregates, a therapeutic agent, a tissue-regenerative agent, a growth factor, a nutraceutical, and combination thereof. In some embodiments, the first liquid component further comprises cells.

In some embodiments, the first liquid component comprises pancreatic islet cells, hepatic cells, neural cells, vascular endothelial cells, thyroid cells, parathyroid cells, adrenal cells, thymic cells, mesenchymal stem cells, and ovarian cells. In some embodiments, the first liquid component comprises cells are selected from the group consisting of pancreatic islet cells, mesenchymal stem cells, parathyroid cells, and thyroid cells. In some embodiments, the first liquid component comprises cells are selected from the group consisting of pancreatic islet cells and parathyroid cells. In some embodiments, the first liquid component comprises pancreatic islet cells. In some embodiments, the first liquid component comprises mesenchymal stem cells genetically modified to express growth factor or coagulation factor VIII. In some embodiments, the first liquid component does not comprises embryonic stem cells. In some embodiments, the first liquid component comprises mesenchymal stem cells.

In some embodiments, the first liquid component further comprises pancreatic islet cells. In some embodiments, the first liquid component further comprises pancreatic islet cells derived from a human or pig. In some embodiments, the first liquid component further comprises pancreatic islet cells derived from a human, adult pig, or neonatal pig. In some embodiments, the first liquid component further comprises about 20,000 to about 40,000 pancreatic islet cells.

The coating may be made by various methods known in the art. In some embodiments, the coating is formed by placing a small amount of the polysaccharide in a container and then contacting the surface of the foam with the solution; and then contacting the solution of polysaccharide with a solution of gel-forming ions. The solution of polysaccharide can also be applied by other methods known in the art, such as dipping, spraying, or draw-down methods. In some embodiments, the coating is formed by contacting the polysaccharide with a self-gelling composition such as those described in U.S. Patent Publication 2006/0159823, published Jul. 20, 2006, which is hereby incorporated by reference in its entirety.

Temperature-sensitive materials may suitably be incorporated in the composite as the composite is suitably formed by a process at or near to ambient temperature. The method of the invention does not need to include a drying step at elevated or reduced temperature for example a freeze drying step although this may be included if desired. The option to avoid drying the composite, especially at elevated temperature, advantageously allows the production of a composite in which the incorporated temperature-sensitive material may be evenly distributed and not deactivated or altered.

The invention further provides a product produced by the method of the invention.

### Uses and Methods for Using the Composites

In a further aspect, the invention provides for the methods and use of a composite produced according to the method of the invention. The composite is particularly suitable for use in medical applications, for example in wound management, tissue engineering and tissue regeneration and cell immobilization.

The uses and methods described herein can be used with any of the composites described herein, products of the processes described herein, or embodiments thereof, including combinations of the described embodiments.

The present invention further provides a method for promoting cell proliferation in the composites described herein comprising washing the composite with a solution, adjusted for osmotic pressure neutral to living cells, and which solution contains gel-forming ions. In some embodiments, the gel-forming ion is selected such that the washing step modifies a mechanical property of the composite. In some embodiments, the solution used to wash the composite contains a salt selected from a calcium salt, a barium salt and a strontium salt in a concentration from about 5 mM to about 200 mM.

The present invention further provides a method for inhibiting cell proliferation comprising forming a composite by a method described herein wherein the first liquid component comprises cells, said polysaccharide being gelled within the pores of the foam wherein the gel-forming ion comprises strontium ion.

The present invention further provides a method for recovery of cells from the composites described herein comprising contacting an aqueous solution comprising a recovery agent with the gel comprising the polysaccharide wherein the said aqueous solution is adjusted to provide an osmotic pressure neutral to living cells. In some embodiments, the recovery agent comprises sodium citrate or other soluble salt of citric acid, sodium EDTA or other soluble salt of EDTA, or hexametaphosphate.

The present invention further provides a method of promoting cell growth comprising implanting a composite described herein, wherein the foam comprises alginate into a human or animal body for the purpose of establishing cell growth.

The present invention further provides a method for preventing adhesion of tissue to adjacent tissue comprising applying to the tissue a composite described herein such that it provides a barrier between the tissue and the adjacent tissue.

The present invention further provides the use of the composites described herein as matrices for cell immobilization and/or proliferation for an in vitro tissue culture application or an in vivo tissue engineering application.

The present invention provides a method of administering one or more composites to a patient in need thereof, comprising implanting said one or more composites in said patient. In some embodiments, the one or more composites are implanted subcutaneously. In some embodiments, three to four composites are implanted into the patient. In some embodiments, the gel of the composite comprises pancreatic islet cells and the patient is in need of treatment of diabetes or regulation of blood glucose levels.

In some embodiments, a therapeutically effective number of cells are implanted. The number of cells needed for the treatment of a specific disorder will vary depending the specific disorder(s) being treated, the size, age, and response pattern of the individual the severity of the disorder(s), the judgment of the attending clinician, the manner of administration, and the purpose of the administration, such as prophylaxis or therapy. The phrase “effective amount” refers to the number of cells that elicits the biological or medicinal response in a tissue, system, animal, individual, patient, or human that is being sought by a researcher, veterinarian, medical doctor or other clinician. The desired biological or medicinal response may include preventing the disorder in an individual (e.g., preventing the disorder in an individual that may be predisposed to the disorder, but does not yet experience or display the pathology or symptomatology of the disease). The desired biological or medicinal response may also include inhibiting the disorder in an individual that is experiencing or displaying the pathology or symptomatology of the disorder (i.e., arresting or slowing further development of the pathology and/or symptomatology). The desired biological or medicinal response may also include ameliorating the disorder in an individual that is experiencing or displaying the pathology or symptomatology of the disease (i.e., reversing the pathology or symptomatology).

One or more composites can be implanted in a patient to reach a therapeutically effective amount of cells. In some embodiments, the gel independently comprises about 5,000 cells or more. In some embodiments, the gel comprises from about 5,000 to about 300,000 cells, about 5,000 to about 200,000 cells, about 5,000 to about 100,000 cells, about 10,000 to about 100,000 cells, about 5,000 to about 60,000 cells, about 10,000 to about 60,000 cells, about 20,000 to about 60,000, or about 20,000 to about 40,000 cells.

The present invention further provides uses of the methods described herein. The present invention further provides a composite described herein for use in a method of treatment of the human or animal body by therapy. In some embodiments, the composite is used in a method of treatment of diabetes or reducing blood glucose levels.

The present invention further provides a kit for implanting one or more composites in a patient in need thereof, comprising one or more composites of the invention. In some embodiments, the kit further comprises instructions for implanting said composites in a patient.

In some embodiments, the composite acts as a carrier for the functional component, releasing the component in use. The composite may be tailored to release the component in a particular manner particularly where the functional component is a pharmaceutical. The “release profile” of the component may be immediate, fast release, controlled release or pulsatile release as desired.

In some embodiments, the composite further comprises an therapeutic agent to provide in vivo controlled release of the active agent into a human or animal body. In some embodiments, the composite further comprises a component for use in managing a wound to provide in vivo wound management.

In some embodiments it is desired to release the entrapped component from the gel. Release may be achieved by physical attrition of the gel, extraction from the gel, or dissolution of the gel, or by simple diffusion or leakage from the gel. The process selected to release the component depends upon the application and the nature of the component to be released and could include application of mechanical or sonic energy, temperature or pH change, or addition of “de-gelling” agents such as chelating agents.

In some embodiments, the composite may be useful in tissue growth and tissue engineering in which functional tissue is created using cells seeded on three dimensional scaffolds that provide a template to guide the growth of new tissue and ensure nutrients reach the cells and waste products are removed. In some embodiments, the composite can be engineered to facilitate the desired ingrowth and undergo degradation in a controllable and predictable manner. For example, when newly developed tissues propagate through the composite, the composite suitably degrades and provides space for new tissue formation. In some embodiments, the composites may be designed to stimulate specific interactions with immobilized cells and/or cells in the area where the composite is implanted, for example, by releasing cell-interacting molecules and growth factors for example for regeneration of bone, nerves, skin and cartilage. In some embodiments, the composite may release growth factors to encourage ingrowth of cells based on a desired geometry and the controlled degradation of the implant allows the regenerated bone tissue to become load bearing. In some embodiments, the composite may be designed to promote or inhibit cell proliferation as appropriate by using calcium ions or strontium ions as the gel-forming ions, respectively.

In some embodiments, cells immobilized in the composite may be implanted into animals wherein the gel and the coating act as an immune barrier and prevents detection by the immune system thereby allowing the implantation of xenografts. In some embodiments, strontium may be used as gel-forming ions when animal cells are desired for implantation (xenografts), since when using this type of artificial organ, it is important that the cells do not grow out of the implanted composite and become exposed to the immune system. In some embodiments, the composite may also be useful to establish cell, tumor and tissue xenografts in animals for, for example, cancer research. Immobilization of multicellular aggregates, such as islets Langerhans, in the composite allows said multicellular aggregates to be implanted into animals or humans without immune rejection and such implanted cell aggregates may then function as an artificial organ producing, for example, insulin.

Cell cultures may be used to manufacture many biological materials, for example enzymes, hormones, immunobiologicals (such as monoclonal antibodies, interleukins, lymphokines) and anticancer agents. Cells can be cultured in composites according to the invention to increase the total number of cells. For example, cells isolated from a patient can be cultured in a composite of the invention to increase the cell number, the cells can then be retrieved from the composite and used in tissue engineering applications. Cell cultures in a composite according to the invention can also be used to explore, characterize and specify cell differentiation and growth to produce tissue like structures. For example, cells are affected by the external stress and modifying the elasticity of the composite (gel/foam) materials may influence gene expression.

Cells produced in vitro in the composite may suitably be recovered from the culture using a recovery agent. Suitable recovery agents include sodium citrate or other soluble salt of citric acid, sodium EDTA or other soluble salt of EDTA, and hexametaphosphate.

Without wishing to be bound by any theory, it is believed that the rigidity of the composite and the gel in which cells are immobilized are important factors for cell growth since it appears that the mechanical properties of the gel regulates proliferation, and differentiation has been observed based on cell type. The rigidity of the gel (as measured, for example, by elastic modulus) on which the cell is attached determines the magnitude of the force generated from the exoskeleton and the extent of cell spreading that ensues. The properties of alginate gels are varied by alginate concentration, saturation of gel-forming ions, and type of gel-forming ions. In addition, alginates can be chemically modified to add peptide sequences for cell adhesion, such as cell adhesion peptide sequences such as the RGD tripeptide.

In some embodiments, the composites may be used in the treatment of the human or animal body to prevent adhesion between tissues. Surgical interventions may cause conglutination or growing together of tissues, e.g. between muscles, between muscles and tendons or nerves or other tissues. To prevent this undesired tissue growth, in some embodiments, an anti-adhesion layer can be inserted between muscles, muscles and tendons or nerves to cover the wound and prevent postoperative adhesion formation during the healing process. In some embodiments, the composites may be formulated for use as an anti-adhesion layer by selection of materials for example a hyalouronate foam in the composite and gelling ions which retards or prevents cell growth and intrusion into the anti-adhesion layer thus avoiding adhesion between tissues during healing. In some embodiments, the composites can be engineered from biodegradable materials which dissolve as the wound heals (by appropriately varying the amount of cross linking ions, type of polymer, polymer concentration) and are degraded or excreted from the body.

Depending upon the formulation properties, in some embodiments, the composite can be formulated to degrade over various periods of time and thereby release immobilized materials such as therapeutic agents or tissue-regenerative agents. A preferred use of the invention is in tissue repair wherein organic or inorganic material can be immobilized within the composite and act as a scaffold for tissue regeneration. One such example would be the inclusion of hydroxyapatite in the gel within the foam and then implanted into a bone defect in order to induce bone regeneration into the foam/gel composite. Another such example would be the inclusion of chemotactic or cell attractant substances within the composite followed by implantation of the composite in a tissue injury site in order to promote tissue regeneration.

When composites are to be used as controlled delivery applications, e.g. of pharmaceuticals, growth factors, nutriceuticals, flavors or fragrances, the mechanical and chemical properties can be modified for appropriate release in the desired environment.

The foam may be shaped prior to or during use as desired. The foam may be secured to a substrate with a fastener, for example a suture, and the soluble polysaccharide then added to the foam to produce the gel and form the composite. As desired, the composite may be formed and then secured to a substrate using a fastener, for example a suture.

In order that the invention disclosed herein may be more efficiently understood, examples are provided below. It should be understood that these examples are for illustrative purposes only and are not to be construed as limiting the invention in any manner.

### GLOSSARY

Albumin Bovine albumin, Fraction V, approx. 99% (A-3059) (Sigma-Aldrich Chemie Gmbh, Steinheim, Germany)
Antibiotic-Antimycotic Antibiotic-Antimycotic solution (0710) GIBCO® (Invitrogen Corp., Grand Island, N.Y., USA)
C₂C₁₂ Mouse myoblast cell line (ATCC # CRL-1772)
CaCl₂ Calcium chloride dihydrate, Ph. Eur. (Riedel-de Haën, Seelze, Germany)
CaCl₂ Calcium chloride dihydrate (1.02382.1000) (Merck KgaA, Darmstadt, Germany)
CaCO₃ Eskal 500, Calcium carbonate, particle size ˜5.2 μm (KSL Staubtechnik, Launingen, Germany)
CaCO₃ HuberCAL 500 Elite, Calcium carbonate, particle size ˜4.2 μm (Huber Engineered Materials, Hamina, Finland)
CaCO₃ HuberCAL 250 Elite, Calcium carbonate, particle size ˜8.7 μm (Huber Engineered Materials, Hamina, Finland)
Calcein Calcein, AM, 1 mg/ml (C3099) (Invitrogen, Molecular Probes, Eugene, Oreg., USA)
Citrate Sodium citrate dihydrate, A.C.S Reagent (Sigma-Aldrich Chemie GmbH, Steinheim, Germay)
FBS Fetal Bovine Serum, GIBCO™ (Sigma-Aldrich Chemie GmbH, Taufkirchen, Germany)
Fluorescent dextran 10 kDa Dextran, fluorescein, 10 000 Mw, anionic (D-1821) (Molecular Probes, Oregon, USA)
Fluorescent dextran 70 kDa Dextran, fluorescein, 70 000 Mw, anionic (D-1822) (Molecular Probes, Oregon, USA)
GDL Glucono δ-lactone (Roquette, Alessandria, Italy)
Glycerine Glycerin, Ph. Eur. (VWR Prolabo, Leuven, Belgium)
Growth medium Dulbeccos's Eagle medium, modified (D-MEM) for Chondrocytes
Chondrocytes (61965-026) GIBCO™ (Sigma-Aldrich Chemie GmbH, Taufkirchen, Germany). Added 10% heat inactivated FBS (20 minutes at 56° C.), 1% Antibiotic-Antimycotic and 1% sodium pyruvate.
Growth medium Dulbeccos's eagle medium, modified (D-MEM) for C₂C₁₂ cells
C₂C₁₂ cells (D-7777) (Sigma Chemical Co, St. Louis, Mo., USA). Added 10% heat inactivated FBS (20 minutes at 56° C.), 3.7 g/l NaHCO₃, 10 ml/l non-essential amino acids, 10 ml/l Penicillin-Streptomycin solution, 1.4 mg/l puromycin and MQ-water.
Growth medium Minimum essential medium eagle (MEM) for MDCK cells (M0643)
MDCK cells (Sigma Chemical Co, St. Louis, Mo., USA). Added 10% heat inactivated FBS (20 minutes at 56° C.), 10 ml/l Penicillin-Streptomycin solution, 2.2 g/l NaHCO₃ and MQ-water.
GDL Glucono δ-lactone (Roquette, Alessandria, Italy)
Glycerine Glycerin, Ph. Eur. (VWR Prolabo, Leuven, Belgium)
Hanks' Hanks' balanced salt solution; Modified; With NaHCO₃; Whithout phenol red, calcium chloride and magnesium chloride (Sigma-Aldrich Chemie GmbH, Steinheim, Germay)
HPMC Pharmacoat 603, Substitution type 2910, Hypromellose USP, (hydroxypropylmethylcellulose) (Shin-Etsu Chemical Co. Ltd., Japan)
Isoton II COULTER® ISOTON® II Diluent (Beckman Coulter, Krefeld, Germany)
Live/Dead test kit Viability/Cytotoxicity kit for animal cells (Invitrogen, Molecular Probes, Eugene, Oreg., USA)
Mannitol D-Mannitol 98% (Sigma-Aldrich Chemie GmbH, Steinheim, Germany)
MDCK Madin Darby Canine Kidney cell line (ATCC #CCL-34)
MQ-water MiliQ water
NaCl Sodium chloride, p.a., (Merck, Darmstadt, Germany)
NaHCO₃ Sodium bicarbonate (Sigma-Aldrich Chemie GmbH, Steinheim, Germany)
Na₂HPO₄ Disodium hydrogen phosphate, art: 30427 (Riedel-de Häen, Seelze, Germany)
Na-triphosphate Sodium triphosphate pentabasic (T5883-500G) (Sigma-Aldrich Chemie Gmbh, Steinheim, Germany)
NOVATACH RGD Peptide coupled PRONOVA UP MVG alginate, batch: CBIFMC01A02122005, sterile filtered and lyophilized. Peptide sequence: GRGDSP. Ratio peptide:alginate 9.11:1
NOVATACH VAPG Peptide coupled PRONOVA UP MVG alginate, batch: CBIFMC02A02122605, sterile filtered and lyophilized. Peptide sequence: VAPG. Ratio peptide:alginate 13.5:1
Penicillin-Streptomycin Penicillin-Streptomycin solution (P0781) (Sigma-Aldrich Chemie GmbH, Steinheim, Germany)
Protanal® LF 200S Sodium alginate, pharma grade, viscosity (1 wt % aqueous solution at 20° C.)=302 mPas (FMC, Philadelphia, Pa., USA)
PRONOVA UP MVG Sodium alginate, batch: 701-256-11, viscosity (1 wt % aqueous solution at 20° C.)=385 mPas (NovaMatrix, Oslo, Norway
PRONOVA UP LVG Sodium alginate, batch: FP-502-04, viscosity (1 wt % aqueous solution at 20° C.)=50 mPas (NovaMatrix, Oslo, Norway
PRONOVA SLG 100 Sterile sodium alginate, viscosity (1 wt % aqueous solution at 20° C.)=166 mPas (NovaMatrix, Oslo, Norway)
PRONOVA SLG 20 Sterile sodium alginate, viscosity (1 wt % aqueous solution at 20° C.)=37.5 mPas (NovaMatrix, Oslo, Norway)
PRONOVA SLM 20 Sterile sodium alginate, viscosity (1 wt % aqueous solution at 20° C.)=9.0 mPas (NovaMatrix, Oslo, Norway)
PRONOVA SLM 20 Sterile sodium alginate, viscosity (1 wt % aqueous solution at 20° C.)=92 mPas (NovaMatrix, Oslo, Norway)
PRONOVA UP LVG Sodium alginate, viscosity (1 wt % aqueous solution at 20° C.)=25 mPas (NovaMatrix, Oslo, Norway)
PRONOVA UP LVG Ultrapure sodium alginate, batch: 221105, viscosity (1 wt % aqueous solution at 20° C.)=35 mPas (Novamatrix, Oslo, Norway)
PRONOVA UP LVG Ultrapure sodium alginate, batch: FP-502-04, viscosity (1 wt % aqueous solution at 20° C.)=50 mPas (Novamatrix, Oslo, Norway)
PRONOVA UP LVG Sodium alginate, viscosity (1 wt % aqueous solution at 20° C.)=92 mPas (NovaMatrix, Oslo, Norway)
PRONOVA UP MVG Ultrapure sodium alginate, batch: FP-310-01, viscosity (1 wt % aqueous solution at 20° C.)=296 mPas (Novamatrix, Oslo, Norway)
PRONOVA UP MVG Ultrapure sodium alginate, batch: FP-312-03, viscosity (1 wt % aqueous solution at 20° C.)=248 mPas (Novamatrix, Oslo, Norway)
PRONOVA UP MVG Ultrapure sodium alginate, batch: 701-256-11, viscosity (1 wt % aqueous solution at 20° C.)=385 mPas (NovaMatrix, Oslo, Norway)
PROTASAN CL 210 (214) Chitosan chloride, batch: 708-783-01, deacetylation: 94.5%, pH=5.3, viscosity of 1% aqueous solution at 20° C.=77 mPas (NovaMatrix, Oslo, Norway)
PROTASAN UP CL 213 Ultrapure chitosan chloride, batch: FP-104-02, viscosity (1 wt % aqueous solution at 20° C.)=74 mPas, degree of deacetylation=86% (NovaMatrix, Oslo, Norway)
Propidium Iodide (P4170) (Sigma Chemical Co., St. Louis, Mo., USA)
Puromycin Puromycin dihydrochloride (P7255) (Sigma Chemical Co., St. Louis, Mo., USA)
Sodium Hyaluronate Pharma grade 80, batch: 17053P, molecular weight: 1.08*10⁶ g/mole (NovaMatrix for Kibun Food Kemifa Co., Ltd., Kamogawa, Japan)
Sodium pyruvate Sodium pyruvate 100 mM solution (S-8636) (Sigma Chemical Co., St. Louis, Mo., USA)
Sorbitol, D(−)sorbitol for biochemistry, dry, 100% (Merck, KGaA, Darmstadt Germany)
Sorbitol special 70% sorbitol solution (SPI Polyols, New Castle, Del., USA)
SrCl₂ Strontium chloride hexahydrate 99% A.C.S. Reagent (Sigma-Aldrich Chemie Gmbh, Steinheim, Germany)
SrCO₃ Strontium carbonate 99.9+% (Sigma-Aldrich Chemie GmbH, Steinheim, Germany)

### EXAMPLE 1

This example shows how the stiffness or the elasticity of the foams is varied as a function of saturation with gel-forming ions and temperature.

The wet foam formulations for the three different foams tested are presented in Table 1. The formulations vary the calcium carbonate such that the calcium ions are sufficient to saturate the gelling sites of the alginate with 66%, 111% and 155%, respectively.

TABLE 1
Wet foam formulations.

| | 66% | 111% | 155% |
|---|---|---|---|
| Ingredient | saturated | saturated | saturated |
| 4% alginate solution | 111.2 | 111.2 | 111.2 |
| (PRONOVA UP MVG, 701-256- | | | |
| 11) | | | |
| Glycerin | 6.0 | 6.0 | 6.0 |
| Sorbitol special | 18.0 | 18.0 | 18.0 |
| HPMC | 3.0 | 3.0 | 3.0 |
| CaCO₃ (HuberCAL 500 Elite) | 0.45 | 0.75 | 1.03 |
| GDL | 1.61 | 2.69 | 3.77 |
| MQ-water | 59.7 | 58.4 | 57.0 |
| Total, Amount in [g] | 200.0 | 200.0 | 200.0 |

An aqueous solution of alginate was prepared and set aside. Calcium carbonate was dispersed in the water (25 grams less than amount shown in Table 1) in a mixing bowl. Glycerin, sorbitol special, the aqueous alginate solution, and HPMC were added to the same bowl and the dispersion was blended with a Hobart kitchen aid mixer equipped with a wire whisk at medium speed for one minute to ensure homogeneity. Mixing continued an additional seven minutes at high speed before adding a freshly mixed GDL solution (i.e., the GDL plus the 25 grams of water) and further mixing at high speed for 1 minute, which gave a resulting wet foam density of 0.25 g/ml (as determined from the weight of wet foam required to fill a 100 ml container). The foams were cast in 2 mm high molds coated with Versi-Dry bench protector with the polyethylene side towards the foam (Nalgene Nunc International, NY, USA) and kept uncovered for 60 minutes at room temperature before drying at 80° C. in a drying oven for 30 minutes. The dried sheets of foams appeared somewhat different as they varied in pore size and thickness. As the gelling rate of the wet foam material is correlated with the saturation with gel-forming ions, coalescence of pores will occur most for the lowest saturated foams.

Circular patches with a diameter of 2.1 cm were stamped out of the dried foam sheets. Some of the foam patches from the 111% saturated foam were autoclaved at 121° C. for 20 minutes.

The foam was prepared for mechanical testing by placing a foam patch in a Petri dish with a diameter of 3.5 cm and adding 4 ml of a model physiological solution (2.5 mM CaCl₂ and 9 mg/ml NaCl). The foam was kept in this solution for 5 minutes before it was transferred to a Bohlin CVO 120 High Resolution Rheometer. The foam was placed between serrated plates (PP 25) with a gap of 500 μm prior to oscillation measurements. Stress sweeps were performed with an applied shear stress from 0.5 Pa to 50 Pa. The frequency was set to 1 Hz. The sweep was performed three times for each foam patch. The elastic modulus, G′, read in the linear viscoelastic region (G′ₗᵢₙ) is reported in Table 2. Tests were performed at two different temperatures. The temperature of the added physiological solution, temperature during swelling, and during the measurements was either 20° C. or 37° C.

TABLE 2
G′ₗᵢₙ of Foam Compositions at two temperatures (n = 3).

| | G′ₗᵢₙ ± SEM at | G′ₗᵢₙ ± SEM at 37° C., |
|---|---|---|
| Foam | 20° C., [Pa] | [Pa] |
| 66% saturated | 762 ± 5 | 736 ± 29 |
| 111% saturated | 2375 ± 52 | 1820 ± 41 |
| 111% saturated | 2469 ± 39 | Not tested |
| (autoclaved) | | |
| 155% saturated | 5374 ± 358* | 3943 ± 195 |

*n = 6
The data show an increase in elastic modulus as a function of increased amount of gel-forming ions. Autoclaving conditions did not seem to affect the foam modulus at 111% saturation.

### EXAMPLE 2

This example shows how gel-forming ions diffuse from the foam to an added alginate solution and thereby induce gelling. The elastic modulus was measured as a function of time after the alginate solution was added to describe the gelling kinetics and the increase in material elasticity. The coating was not added in this study.

A composite was prepared from dry foam disks (2.1 cm in diameter) from the same 66% and 111% saturated foams prepared in example 1 and 250 μl 1% PRONOVA UP LVG (99 mPas) by uniformly distributing the solution by dropwise addition from a pipette onto the top surface of the foam. All of the alginate solution added was absorbed and it filled pores through the foam. The calcium ions present in the 66% and the 111% saturated foams saturate 43% and 71% of the total amount of alginate in the composite. The composite was transferred to the rheometer 5 minutes after addition of the alginate solution. The gap was set to 300 μm and the frequency and strain were kept constant at 1 Hz and 0.001 respectively. The elasticity modulus, G, of the composite as a function of time is presented in Table 3.

TABLE 3
G as a function of time after addition of alginate solution to the foams

G ± SEM at 20° C., [Pa]

| Time, [min] | 66% saturated foam | 111% saturated foam |
|---|---|---|
| 6 | 828 | 1787 |
| 15 | 858 | 2931 |
| 30 | 893 | 3499 |
| 45 | 919 | 3863 |
| 60 | 942 | 4020 |
| 75 | 970 | 4059 |
| 90 | 997 | 3986 |
| 105 | 1067 | 4027 |

The results show an increase in elastic modulus as a function of time for both foams, which indicate the diffusion of gel-forming ions from the alginate foam to the added alginate solution and the resulting gelling. The values of G increase fast and reach a plateau after 60 minutes for the 111% saturated foam. For the 66% saturated foam the G values are lower, increase more slowly and the results indicate that the diffusion was not completed within the 105 minutes with testing.

### EXAMPLE 3

This example shows how the elasticity can be modified with use of foams containing varying amounts gel-forming ions and by adding alginate solutions with different G-content and molecular weight. The coating was not added in this study.

The foams tested was the 111% saturated foam and the 111% saturated foam which had been autoclaved from example 1, and a new 155% saturated foam prepared as in example 1 except using a blend of calcium carbonate where half of the CaCO₃ was replaced with a CaCO₃ with larger particle diameter (HuberCAL 250 Elite, 8.7 μm). This foam had somewhat larger pores than the 155% saturated foam from example 1 and was able to absorb more alginate solution with a higher viscosity. The wet density of the 155% saturated foam was 0.24 g/ml. Foam disks (diameter 2.1 cm) as example 1 were placed in petri dishes and added on the top surface 250 μl alginate solution with use of a pipette. All of the alginate solution added was absorbed and it filled the pores through the foams. The dishes containing foams with alginate solution added were kept at room temperature for 60 minutes and then 4 ml of the model physiological solution was added. After 5 minutes, the disc was transferred to the rheometer and a stress sweep was performed as described in example 1 except the gap was set to 300 μm. The measured G′ₗᵢₙ for these samples are presented in Table 4. The G-content of the PRONOVA UP LVG is about 67% (high-G) whereas PRONOVA SLM 20 contains about 43% G (high-M). The alginate samples with the lower viscosity were prepared by degradation of the molecular weight of the alginate by autoclaving 1% and 2% solutions of the PRONOVA SLM 20 and PRONOVA UP LVG, respectively, for 20 minutes at 121° C.

TABLE 4
G′ₗᵢₙ of composites with varying alginate concentration of the added
alginate solution for two molecular weights (1% viscosity) of high
M alginate

| | | | G′ₗᵢₙ ± SEM at | G′ₗᵢₙ ± SEM at |
|---|---|---|---|---|
| | | | 20° C., [Pa] | 20° C., [Pa] |
| | PRONOVA | Overall | for composites | for composites |
| Foam | SLM 20 | composite | with SLM 20 | with SLM 20 |
| saturation | alginate | saturation | visc = 9 mPas | visc = 92 mPas |
| 111% | 1.0% | 82% | 3373 ± 24 | 4063 ± 208 |
| 111% | 0.75% | 88% | 2856 ± 139* | 3846 ± 196 |
| 111% | 0.5% | 94% | 2720 ± 164* | 3498 ± 168 |
| 111% | 0.25% | 102% | 2963 ± 38 | 3089 ± 231 |

(* n = 5)

TABLE 5
G′ₗᵢₙ of composites with varying concentration of the added alginate
solution for two molecular weights (1% viscosity) of high G alginate

| | | | G′ₗᵢₙ ± SEM at | G′ₗᵢₙ ± SEM at |
|---|---|---|---|---|
| | PRONOVA | Overall | 20° C., [Pa] | 20° C., [Pa] |
| Foam | UP LVG | composite | for LVG visc = | For LVG |
| saturation | alginate | saturation | 25 mPas | visc = 99 mPas |
| 155% | 1.0% | 98% | 5037 ± 209 | not tested |
| 155% | 0.5% | 120% | 3792 ± 185 | not tested |
| 111% | 1.2% | 67% | 4344 ± 44 | 5060 ± 259* |
| 111% | 1.0% | 71% | 3870 ± 197 | 4740 ± 71 |
| 111% | 0.75% | 78% | 3485 ± 114 | 3522 ± 115 |
| 111% | 0.5% | 87% | 3189 ± 61 | 3147 ± 70 |
| 111% | 0.25% | 97% | 3241 ± 174 | 2786 ± 100 |
| 111% | 1.0% | 71% | not tested | 4768 ± 147 |
| Autoclaved | | | | |

(*n = 5)

The data show that the composites with the highest elastic modulus at both 1.0% and 0.5% added alginate were 155% saturated foams with the added high-G alginate. The molecular weight of the alginate is of importance for both high-G and high-M alginates as the elastic modulus G′ₗᵢₙ decreased with decreasing viscosities, except for alginate concentrations of 0.75% and below for the high-G alginate where similar results were obtained. In general, the elastic modulus G′ₗᵢₙ decreased as the concentration of the alginate added decreased for both the 155% and the 111% saturated foams, and for the four different alginates. Exceptions were observed for the low viscosity alginates at lowest concentrations. The elastic modulus G′ₗᵢₙ in of the composite was not altered by autoclaving the foam.

### EXAMPLE 4

This example shows how the elasticity of the composite material is influenced by washing the composite in a solution containing additional gel-forming ions. The coating was not added in this study.

Dry foam disks (diameter about 2.1 cm) from the 111% saturated foam in example 1 were placed in Petri dishes and 250 microliters 1% PRONOVA UP LVG (99 mPas) was added to the top surface of the foams and held at room temperature 60 minutes. Then the composites were incubated in 4 milliliters of either a 50 mM solution of CaCl₂ or a 50 mM solution of SrCl₂. After 5 minutes was the solution containing extra gel-forming ions was replaced with model physiological solution and after another 5 minutes was the G in measured as described in example 1. Another sample which did not receive any added extra gel-forming ions had the model physiological solution added 60 minutes after the alginate was added, and the G′ₗᵢₙ was measured after 5 minutes swelling. The results are presented in Table 6.

TABLE 6
Modulus G′ₗᵢₙ of composites prepared with and without
washing in a solution containing extra gel-forming ions (n = 3).

| Wash | G′ₗᵢₙ ± SEM at 20° C., [Pa] |
|---|---|
| 50 mM CaCl₂ | 13 100 ± 700 |
| 50 mM SrCl₂ | 15 900 ± 600 |
| Not washed | 2 700 ± 100 |

The data show more than four to nearly six times increase in elastic modulus G′ₗᵢₙ by washing the composites with a solution containing calcium or strontium ions respectively. A higher value was obtained for the composites washed in the solution containing strontium ions which created a more rigid gel network than the composite washed with calcium ions.

### EXAMPLE 5

This example shows how proliferation and viability of MDCK (Madin Darby Canine Kidney) cells immobilized in alginate are influenced by varying the calcium saturation of the alginate foam, and the effect of adding additional gel-forming ions to the composite after cell immobilization. The coating was not added in this study.

Two different alginate foams were prepared with calcium ions sufficient to saturate 100% and 200% of the gelling sites of the alginate. The wet foam formulations are presented in Table 7.

TABLE 7
Wet foam formulations.

| Ingredient | 100% Saturated foam | 200% Saturated foam |
|---|---|---|
| 4% alginate solution | 125 | 125 |
| (PROTANAL LF | | |
| 200S) | | |
| Glycerin | 6.0 | 6.0 |
| Sorbitol special | 18.0 | 18.0 |
| HPMC | 3.0 | 3.0 |
| CaCO₃ (Eskal 500) | 0.76 | 1.52 |
| GDL | 2.66 | 5.32 |
| Deionized water | 44.6 | 41.2 |
| Total [amount in g] | 200.0 | 200.0 |

An aqueous solution of alginate was prepared. Then the CaCO₃ was dispersed in the water as listed above, except for 25 g, in a mixing bowl. Glycerin, sorbitol special, the aqueous alginate solution and HPMC were added to the same bowl and the dispersion was blended with a Hobart kitchen aid mixer equipped with a wire whisk at medium speed for 1 minute to ensure homogeneity. For the 100% saturated foam, mixing continued for 3 minutes at high speed. GDL was then dissolved in the remaining 25 g of water and added to the wet foam. The dispersion was further mixed at high speed for 30 seconds. The resulting wet density of the 100% foam was 0.23 g/ml. For the 200% saturated foam, the high speed mixing time was 3.5 minutes before GDL addition and then additionally 15 seconds of high speed mixing. The 200% foam had a wet density of 0.26 g/ml. Both foams were cast in 1 mm high Teflon coated molds and kept uncovered for 15 minutes at room temperature before drying at 80° C. in a drying oven for 30 minutes.

Disks (diameter=3.6 cm) were cut from the dried foam sheets with a scalpel and packed separately. The foam disks were then autoclaved at 121° C. for 20 minutes.

Sterile alginate (PRONOVA SLG 100) was dissolved in cell growth medium (MEM) to a 1% alginate solution. The alginate solution and a suspension of MDCK cells in growth medium were blended to a final concentration of 0.8% alginate and 200 000 cells/ml. The alginate foam discs were transferred to wells in a 6 wells plate (Nunclon®, Nalgene Nunc International), where they closely fit the well size. 1.0 ml of the alginate cell suspension was distributed drop-wise with a pipette to each of the foams and the alginate composites were incubated at 37° C. for 20 minutes. The calcium ions present in the 100% and 200% saturated foam were sufficient to saturate 67% and 133% of the gelling sites of the total amount of alginate, respectively. Half of the samples were then given a washing post treatment by adding them to about 5 ml of an aqueous solution containing 50 mM CaCl₂ and 104 mM NaCl. After 10 minutes, the salt solution was replaced with cell growth medium. To the remaining samples cell growth medium were added after the 20 minutes of incubation. The alginate composites with the immobilized cells were kept at 37° C. and growth medium were changed three times a week.

Quantification of cell proliferation and viability were measured after different times after immobilization. The immobilized cells were isolated by transferring the alginate composites to centrifuge tubes containing about 8 ml isotonic citrate solution (50 mM trisodiumcitrate dihydrate and 104 mM NaCl). The tubes were regularly gently turned until the composite was dissolved within about 2-10 minutes, and then centrifuged at 13 000 rpm for 5 minutes. The supernatants were poured off and the pellets containing the cells were re-suspended in 1.0 ml 250 mM mannitol. Three samples of each of 100 microliters, 80 microliters, and 50 microliters of the re-suspended pellets were then transferred to wells in a 96 wells plate (Nunclon®, Nalgene Nunc International). Then zero, 20 microliters, and 50 microliter of mannitol respectively were added (i.e., to fill each well to a total of 100 microliters) and then a further 100 microliters of live/dead reagent. The live/dead reagent was made from 5 ml mannitol solution (250 mM), 20 microliters ethidium solution (2 mM) and 5 μl calcein solution (4 mM). Standard curves were prepared from viable and ethanol fixed cells within the range of 0-10⁶ cells.

The cell proliferation and viability were measured with use of Cytofluor microplate reader. The filters used for Calcein were 485 nm (excitation) and 530 nm (emission), and for Ethidium were 530 nm (excitation) and 620 nm (emission).

TABLE 8
Cell viability and proliferation as a function of time from varying
composites (n = 3, ± SEM).

| | | | | Dead |
|---|---|---|---|---|
| Sample type | Age | Total cell count | Normalized | cells, % |
| 100% unwashed | 1 week | 231 800 ± 10,200 | 1.2 ± 0.1 | 9 ± 2 |
| | 3 week | 130 300 ± 28 700 | 0.65 ± 0.14 | 63 ± 18 |
| | 5 week | 224 700 ± 121 000 | 1.1 ± 0.6 | 45 ± 23 |
| | 7 week | 196 800 ± 28 400 | 1.0 ± 0.1 | 55 ± 9 |
| 100%, washed | 1 week | 189 600 ± 5 100 | 0.95 ± 0.03 | 7 ± 2 |
| | 3 week | 304 900 ± 31 500 | 1.5 ± 0.2 | 37 ± 4 |
| | 5 week | 322 200 ± 82 800 | 1.6 ± 0.4 | 39 ± 11 |
| | 7 week | 660 300 ± 394 600 | 3.3 ± 2.0 | 40 ± 8 |
| 200% unwashed | 1 week | 212 600 ± 34 700 | 1.1 ± 0.2 | 8 ± 3 |
| | 3 week | 258 300 ± 62 500 | 1.3 ± 0.3 | 43 ± 4 |
| | 5 week | 283 400 ± 73 300 | 1.4 ± 0.4 | 44 ± 6 |
| | 7 week | 364 800 ± 22 300 | 1.8 ± 0.1 | 60 ± 2 |
| 200%, washed | 1 week | 255 600 ± 48 900 | 1.3 ± 0.2 | 8 ± 2 |
| | 3 week | 712 800 ± 292 600 | 3.6 ± 1.5 | 18 ± 2 |
| | 5 week | 485 600 ± 217 400 | 2.4 ± 1.1 | 28 ± 5 |
| | 7 week | 663 600 ± 176 500 | 3.3 ± 0.9 | 42 ± 5 |

The data in Table 8 show that the washing step adding extra calcium ions (which provides a more rigid gel network) promotes cell proliferation. As the number of cells increased over time, decreased cell viability was observed.

Investigating the composites in a fluorescence microscope after soaking them in the live/dead reagent showed that the washed composites samples had more cells spreading out.

### EXAMPLE 6

This example shows how proliferation and viability of fast growing myoblast cells from mouse (C₂C₁₂ cells) are affected by the washing step after cell immobilization and the effect of the type of gel-forming ions in the washing solutions. The coating was not added in this study.

An alginate foam was made with calcium as gel-forming ions, sufficient to saturate the alginate by 155%. The wet foam formulation is presented in Table 9.

TABLE 9
Wet foam Formulation.

| Ingredient | Amount, [g] |
|---|---|
| 4% alginate solution | 113.0 |
| (PRONOVA UP MVG, FP-312-03) | |
| Glycerin | 6.0 |
| Sorbitol special | 18.0 |
| HPMC | 3.0 |
| CaCO₃ (HuberCAL 500 Elite) | 1.05 |
| GDL | 3.77 |
| MQ-water | 57.0 |

The wet foam was prepared as described in Example 5, except high speed mixing for 7 minutes before the addition of GDL dissolved in 30 g of the total water followed by an additional 30 seconds high speed mixing. The resulting wet foam density was 0.29 g/ml and the foam was cast in a 2 mm deep mold coated with Versi-Dry bench protector with the polyethylene side towards the foam. The foam was then kept uncovered at ambient temperature for 1 hour before it was dried in a drying oven at 80° C. for 30 minutes.

Disks (diameter=2.1 cm) were stamped out from the dried foam sheets and packed separately. The foam disks were then autoclaved at 121° C. for 20 minutes.

The sterile alginate foams were transferred to wells in a 12 well plate (Nunclon®, Nalgene Nunc International), where they closely fit the well size. 300 μl of a 1% alginate solution (PRONOVA SLG 20) containing 25,000 cells were distributed drop-wise with a pipette to the foam. The foams were then incubated for 20 minutes at 37° C. The calcium ions present in the foam were sufficient to saturate the total amount of alginate in the composite by 97%. Growth medium (D-MEM) was added to one-third of the foams. About 2 milliliters of an isotonic calcium solution (50 mM CaCl₂ and 250 mM mannitol) was added to half of the remaining foams, while the other half of the remaining foams received an isotonic strontium solution (50 mM SrCl₂ and 250 mM mannitol). The gelling solutions were replaced with growth medium after about 2-5 minutes. The foams with the immobilized cells were kept at 37° C. and growth medium was changed three times a week.

Quantification of cell proliferation and viability were measured twice, at day 1 and at 10 weeks after immobilization (FIG. 2). The immobilized cells were isolated as described in Example 5, except different de-gelling solutions were used for the day 1 and 10 week tests. The foams tested 1 day after cell immobilization were dissolved in 10 ml of a solution containing 50 mM citrate and 250 mM mannitol. The foams tested 10 weeks after cell immobilization were dissolved in 10 ml of Hanks' solution containing added 50 mM citrate. The recovered cell pellets were dispersed in 1 ml of Live/dead reagent (made from 4 ml Isoton II, 1 ml propidium iodide (85 μg/ml) and 20 μl calcein (1 mg/ml). Two drops were added a Bürker counting chamber for cell counting in a fluorescence microscope, while the rest of the cell dispersion were filtered through a 60 μm nylon mesh and then five minutes after resuspension, the cells were analyzed with use of a Coulter EPICS Elite flow cytometer.

TABLE 10
Proliferation and viability of C₂C₁₂ cells in three different
composites presented as the mean of 3 or 4 composites ± SEM.

| | | | Dead |
|---|---|---|---|
| | Cell proliferation, | Cell proliferation, | Cells, |
| | total number of cells | normalized, [%] | [%] |
| Foam without wash | | | |
| 1 day | 19,900 ± 1,600 | 0.8 ± 0.1 | 28 ± 3 |
| 10 weeks | 216,300 ± 5,200 | 8.7 ± 0.2 | 70 ± 6 |
| Foam with CaCl₂ wash | | | |
| 1 day | 25,500 ± 1,600 | 1.0 ± 0.1 | 21 ± 4 |
| 10 weeks | 349,000 ± 66,400 | 14 ± 3 | 72 ± 4 |
| Foam with SrCl₂ wash | | | |
| 1 day | 14,400 ± 1,200 | 0.6 ± 0.1 | 32 ± 0 |
| 10 weeks | 117,000 ± 11,500 | 4.7 ± 0.5 | 72 ± 6 |

The results show an increase of the total cell number in all three composites. The proliferation of C₂C₁₂ cells is most highly promoted for cells immobilized in the composites that were washed with a solution containing additional calcium ions after cell immobilization. The slowest growing cells were those cells immobilized in the composites which were washed with a solution containing strontium ions after cell immobilization.

Investigating the composites in a fluorescence microscope showed that the cells immobilized in the composites washed with calcium ions were stretched out and grew both on and through the structure. The cells immobilized in the composites washed with strontium ions were visible as single cells or small clusters.

### EXAMPLE 7

This example shows how cell proliferation and viability of human chondrocytes are affected by varying the source of gel-forming ions in the alginate foam, the washing step after cell immobilization and the effect of the different gel-forming ions in the washing solutions. The coating was not added in this study.

Alginate foams were made with either calcium or strontium as the gelling ion sufficient to saturate the alginate by 155% and 105% respectively. The wet foam formulations are presented in Table 11.

TABLE 11
Wet Foam Formulation.

| Ingredient | Ca-foam | Sr-foam |
|---|---|---|
| 4% alginate solution | 107.8 | 107.8 |
| (PRONOVA UP MVG, FP-310-01) | | |
| Glycerin | 6.0 | 6.0 |
| Sorbitol special | 18.0 | 18.0 |
| HPMC | 3.0 | 3.0 |
| CaCO₃ (HuberCAL 500 Elite) | 1.05 | 0 |
| SrCO₂ | 0 | 1.05 |
| GDL | 3.77 | 3.77 |
| Deionized water | 57.0 | 57.0 |
| Total, Amount in [g] | 200.0 | 200.0 |

The wet foams were prepared as described in Example 6, except using 8 minutes with high speed mixing before addition of GDL dissolved in 30 g water of the total water and using 45 seconds of final high speed mixing. The resulting wet foam densities were 0.30 g/ml and the foams were cast in 2 mm deep molds coated with Versi-Dry bench protector. The foams were then kept uncovered at ambient temperature for 1 hour before they were dried in a drying oven at 80° C. for 30 minutes.

Sterile alginate foam disc preparation and addition of cells to the foams were done as described in Example 7 except that 300 μl of a 1% alginate solution (PRONOVA SLG 20) with 195,000 cells/ml was added to the foams. The gel-forming ions present were sufficient to saturate the total of G-monomers in the alginates by 97% and 63% for the foam gelled with calcium ions and the foam gelled with strontium ion respectively Quantification of cell proliferation and viability were measured twice, at 2 weeks and 11 weeks after immobilization of cells in the alginate foams gelled with Calcium ions (Ca-foams), and once after 13 weeks for the alginate foams gelled with Strontium ions (Sr-foams) and such data are shown in Table 12. The immobilized cells were isolated as described in Example 6, except 15 ml degelling solution (Hanks with 50 mM citrate) were used for the Sr-foams. The sample preparation for cell quantification and use of the flow cytometer were as described in Example 6.

TABLE 12
Proliferation and viability of Chondrocytes in six different
composites presented as the mean of 3 or 4 composites ± SEM.

| | | | | Nor- | |
|---|---|---|---|---|---|
| | | | | malized | dead |
| Foam | Wash | Time | Total cell count | cell count | cells, % |
| Ca-foam | none | 2 weeks | 36,800 ± 3,000 | 0.6 ± 0.1 | 28 ± 4 |
| Ca-foam | none | 11 weeks | 65,400 ± 4,200 | 1.1 ± 0.1 | 50 ± 4 |
| Ca-foam | CaCl₂ | 2 weeks | 50,600 ± 1,800 | 0.9 ± 0.1 | 22 ± 2 |
| Ca-foam | CaCl₂ | 11 weeks | 132,700 ± 12,900 | 2.3 ± 0.4 | 64 ± 5 |
| Ca-foam | SrCl₂ | 2 weeks | 32 800 ± 2,500 | 0.6 ± 0.1 | 27 ± 2 |
| Ca-foam | SrCl₂ | 11 weeks | 35 400 ± 4,800 | 0.6 ± 0.2 | 36 ± 2 |
| Sr-foam | none | 13 weeks | 40,800 ± 2,100 | 0.7 ± 0.1 | 48 ± 2 |
| Sr-foam | CaCl₂ | 13 weeks | 89,700 ± 11,600 | 1.5 ± 0.1 | 63 ± 5 |
| Sr-foam | SrCl₂ | 13 weeks | 52,700 ± 1,800 | 0.9 ± 0.1 | 34 ± 3 |

The results described in Table 12 show that the alginate matrices in composites washed with calcium ion solution had at least twice as many cells after 11 weeks as the other composites. This indicates promoted cell proliferation due to additional calcium ion and/or its effect of providing a more rigid gel matrix. The results also show an inhibited cell proliferation for the cells immobilized in matrixes in composites washed in strontium ion solution. The similar trends were observed for the strontium foams. Strontium foams washed with calcium ion showed the most cell growth of the strontium foam series. The strontium foams without washing or washed with the strontium containing solution showed little or no increase in total cell number. Increased cell death was observed over time as the cells proliferated. Highest percentages of viable cells were observed in the composites containing the slowest growing cells. Investigating the composites in a fluorescence microscope showed that the cells immobilized in the calcium washed composites stretched out and grew both on and through the structure. The cells immobilized in the composites washed with strontium ions were visible as single cells or small clusters.

### EXAMPLE 8

The alginate foams tested was the same as presented in Example 1 and had calcium incorporated sufficient to saturate 111% of the gelling sites of the alginate. Foam discs with a diameter of 1.0 cm were stamped out with a cork borer. An 1.1% aqueous alginate solution was made from PRONOVA UP LVG (FP-502-04). This alginate solution was diluted with MQ-water and a solution with fluorescent dextran (6.25 mg/ml) to give four different solutions varying in alginate concentration and type of fluorescent dextran as presented in Table 13. 80 μl of a solution containing alginate and fluorescent dextran was pipetted onto an alginate foam disc and after 10 minutes the solution was fully absorbed into the foam. The calcium present in the alginate foam was enough to saturate the gelling sites in the total amount alginate in the solution and the foam by 62%. The amount of fluorescent dextran added to each of the foam discs was 45.63 μg. The foam discs were kept in room temperature only covered with alumina foil to avoid light. Each of the foam discs was then separately transferred into a tube containing 10 ml Hanks'. The tubes were horizontally stirred at approx. 20 rpm and samples of 100 μl were collected for quantification of fluorescent dextran that might have leaked out of the composite. The concentration of fluorescent dextran in the collected samples and standard solutions (fluorescent dextran 10 kDa and −70 kDa diluted in Hanks') were analyzed with use of Cytofluor microplate reader. The filters used were 485 nm (excitation, band width 20 nm) and 530 nm (emission, band width 25 nm). The standard curves were made from both types of fluorescent dextrans with five parallels in the range 0 mg/ml to 0.01 mg/ml. The fitting curves gave correlation coefficients R²=0.998 and R²=0.979 for 10 kDa and 70 kDa respectively.

Samples were collected 5-, 15-, 30-, 45-, 60-, 90-, 120- and 150 minutes after the discs were transferred into Hanks' solution. After 150 minutes the measured values reached a plateau. With use of non-linear fit curves described by f(t)=100−100e^{-kt} (k: rate constant, t: time), and calculation programs in GraFit Workspace were the half times determined. The results are presented in table 13. The results show a significant difference in release rate between the two molecular weights of fluorescent dextran. The results also indicate a faster release of both dextrans immobilized in lower concentrations of alginate.

TABLE 13
Solutions added alginate foam discs (V = 80 μl/disc, n = 3).

| Solution | Alginate | Fluorescent dextran, | Half time, |
|---|---|---|---|
| no. | concentration, [%] | molecular weight, [kDa] | [min] ± SD |
| 1 | 1.0 | 10 | 16.9 ± 1.3 |
| 2 | 0.5 | 10 | 15.4 ± 1.3 |
| 3 | 1.0 | 70 | 22.0 ± 1.3 |
| 4 | 0.5 | 70 | 21.2 ± 1.5 |

### EXAMPLE 9

The alginate foam used in this example was added calcium sufficient to saturate 125% of the alginate. The wet foam formulation is presented in table 14.

TABLE 14
Wet foam formulation.

| Ingredient | Amount, [g] |
|---|---|
| 4% alginate solution | 106.8 |
| (PRONOVA UP MVG, FP-311-01) | |
| Glycerin | 6.0 |
| Sorbitol (dry) | 13.9 |
| HPMC | 3.0 |
| CaCO₃ (HuberCal 500 Elite) | 0.85 |
| GDL | 3.01 |
| MQ-water | 66.4 |
| Total | 200.0 |

The foam was made as described in Example 1, except that the wet density was 0.24 g/ml and the foam was cast in a 2 mm high mold coated with Teflon. The equipment used were either depyrogenized by heat treatment at 250° C. for 4 hours or washed in 1 M NaOH. The dried foam was sterilized by gamma-irradiation (dose: 29.5 kGy).

Sterile foam disks (diameter=2.1 cm) were stamped out with use of a cork borer and transferred to wells in a 12 well plate. Table 15 presents three different blends of cells (C₂C₁₂) and alginate that were prepared. The cells were suspended in growth medium (DMEM) and quantified with use of a Bürker cell counting chamber. Regular alginate (PRONOVA SLG 20, batch: 221105) was dissolved in DMEM, whereas peptide coupled alginates (NOVATACH VAPG and NOVATACH RGD) were dissolved in 250 mM mannitol. The densities of peptide coupled to the alginates were measured to be 0.045 μmole/mg solid and 0.031 μmole/mg solid for NOVATACH VAPG and NOVATACH RGD respectively (measured by amino acid method).

TABLE 15
Suspensions for immobilization of cells.

| | Suspension 1 | Suspension 2 | Suspension 3 |
|---|---|---|---|
| Material | [ml] | [ml] | [ml] |
| Cells (1 056 667 cells/ml) | 0.43 | 0.43 | 0.43 |
| Alginate solution (2%, w/v) | 2.25 | 1.75 | 1.53 |
| DMEM | 1.82 | 1.33 | 1.10 |
| NOVATACH VAPG (1%, | — | 1.00 | — |
| w/v) | | | |
| NOVATACH RGD (1%, | — | — | 1.45 |
| w/v) | | | |
| Total | 4.50 | 4.50 | 4.50 |

Each suspension has a total alginate concentration of 1.0%. 250 μl of the suspensions presented in table 15 was added each foam disk, different suspensions to different disks. The calcium in the foam is sufficient to saturate the G monomers in the total amount of alginate with 77%. The peptide concentration in each disk was 0.025 μmole and amount of cells was 25 000 per disk. The suspension was dripped onto the foam with use of a pipette. The foam absorbed all the added solution and the thickness after hydration was about 1.2 mm. The foams were after addition of cell suspension transferred to an incubator and kept at 37° C. for 20 minutes. Then half of the foam disks were added about 2.5 ml DMEM whereas the other half were added about 2.5 ml of an isotonic solution of 50 mM CaCl₂ and 250 mM mannitol. After about five minutes the foams added the calcium containing solution got this solution replaced with DMEM.

After two days the cells were isolated as described in Example 5, except that the foam disks were dissolved in a solution containing 50 mM trisodiumcitrate and 250 mM mannitol. The pellets of cells after centrifugation were resuspended in 600 μl 250 mM mannitol. Three samples of each of 100 μl and 80 μl of the re-suspended pellets were then transferred to wells in a 96 wells plate. Then zero and 20 μl of mannitol respectively were added (i.e., to fill each well to a total of 100 μl) and then a further 100 μl of live/dead reagent. The live/dead reagent was made from 5 ml mannitol solution (250 mM), 20 μl ethidium solution (2 mM) and 5 μl calcein solution (4 mM). Standard curves were prepared from viable and ethanol fixed cells within the range of 0-10⁵ cells. The fitting curves gave correlation coefficients R²=0.988 and R²=0.984 for viable and dead cells respectively.

The quantification of viable cells isolated from each foam disk was performed with use of Cytofluor microplate reader as described in Example 5. The results are presented in table 16. The signals for dead cells were about the blank value for all samples so these data are not shown.

TABLE 16
Cell viability and proliferation as a function of time for
the different composites. (n = 3, ± SEM)

| | Calcium wash after | |
|---|---|---|
| Foam | cell immobilization | Viable cells |
| Suspension 1 | No | 19 400 ± 1 300 |
| Suspension 1 | Yes | 20 200 ± 980 |
| Suspension 2 | No | 31 200 ± 1 900 |
| Suspension 2 | Yes | 20 100 ± 1 000 |
| Suspension 3 | No | 60 800 ± 1 000 |
| Suspension 3 | Yes | 46 300 ± 500 |

### EXAMPLE 10

This example presents a method for producing chitosan foams and their characteristics related to density and absorption.

An aqueous solution containing 4% chitosan salt was prepared using PROTASAN CL 210 (214). 77.0 g MQ-water and 14.0 g sorbitol (dry) were added a mixing bowl and the sorbitol were dissolved by gently swirling the bowl. 100 g of the chitosan solution, 6.0 g glycerin and 3.0 g HPMC were added to the same mixing bowl. The dispersion was blended with a Hobart kitchen aid mixer equipped with a wire whisk at medium speed for one minute to ensure homogeneity. The mixing continued at high speed for 2.5 minutes. The wet density was measured to be 0.23 g/ml (determined from the weight of wet foam required to fill a 100 ml container). The wet foam was cast in 2 mm and 4 mm high molds coated with Teflon and then placed in a drying oven at 80° C. for 30 minutes and 60 minutes, respectively.

Another foam was made by the procedure as above, but the wet foam was molded in a 8 mm deep mold. The foam was dried at 80° C. for 1 hour and then 3 hours at 40° C.

The resulting dry foams were flexible and soft with an open pore network. When water was added to the foam it was immediately absorbed and the foam expanded significantly. The hydrated foam retained its shape, but was relatively weak in that the wet foam could not be transferred in one piece by lifting it from one corner. Compressing the dry foam before hydration did not noticeably affect the foam's absorbency rate or absorption capacity.

To measure the absorption capacity foam pieces were cut at 3.5 cm by 3.5 cm with use of a scalpel. A foam piece was weighted and placed on a mesh (diameter 0.71 mm) and Hanks' Balanced Salt Solution, as a model physiological solution, was added using a pipette. Excess liquid was added and the foams turned transparent. When no dripping from the foam piece was observed, the weight of the wet foam was measured. The dry density and the absorption capacity for the three different foams were measured, and the results are presented in table 17

TABLE 17
Dry density and absorption capacity of a model physiological solution
of chitosan foams of different thickness (n = 3, ±SD).

| Thickness | | | | | Absorption, |
|---|---|---|---|---|---|
| foam before | Thickness | Weight dry | | | [g Hanks' |
| drying, | dry foam, | foam, 3.5 cm | Dry density | Weight wet | absorbed/g |
| [mm] | [mm] | by 3.5 cm, [g] | [g/cm³] | foam, [g] | foam] |
| 2 | 1.95 | 0.101 ± 0.002 | 0.042 ± 0.001 | 2.02 ± 0.04 | 19.0 ± 0.1 |
| 4 | 3.20 | 0.164 ± 0.003 | 0.042 ± 0.001 | 3.20 ± 0.12 | 18.5 ± 0.8 |
| 8 | 5.50 | 0.390 ± 0.013 | 0.058 ± 0.002 | 6.76 ± 0.12 | 16.4 ± 0.3 |

### EXAMPLE 11

This example presents a two-layer foam material made comprising alginate foam as the first layer and chitosan foam as a second layer attached to the alginate foam. This type of composite may be used to modify integrity, strength, biodegradation and absorption capacity of the chitosan foam.

An alginate foam was made by first preparing an aqueous solution containing 4% alginate (PRONOVA UP MVG). 111.2 g of the alginate solution was transferred to a mixing bowl. To the same bowl 6.0 g glycerin, 18.0 g sorbitol special, 3.0 g HPMC, 0.85 g CaCO₃ (sufficient to saturate the guluronic residues in the alginate with 125%) and 33.3 g MQ-water were added. The dispersion was blended with a Hobart kitchen aid mixer equipped with a wire whisk at medium speed for 1 minute and 30 seconds to ensure homogeneity. The mixing continued at high speed for 7 minutes before a freshly mixed GDL solution of 2.69 g GDL and 25.0 g MQ-water was added. The mixing continued at high speed for 1 minute, which resulted in a foam with a wet density of 0.23 g/ml. The wet foam was cast in 4 mm and 2 mm high molds coated with Versi-Dry bench protector with the polyethylene side towards the foam (Nalgene Nunc International, NY, USA) and kept uncovered for 60 minutes at room temperature.

Then wet chitosan foam was added on top of the gelled wet alginate foams as layers of 2 mm and 4 mm (by increasing the mold height) to the top of the 2 mm and 4 mm thick gelled alginate foams, respectively. The chitosan foam was made as described in Example 10 except 18.0 g sorbitol special was used in place of dry sorbitol and 73.0 g MQ-water was added for this foam. The mixing time at medium speed was 2 minutes and then 3 minutes of high speed mixing, which resulted in a foam with a wet density of 0.22 g/ml. The molds with the two-layered foams were then placed in a drying oven at 80° C. for 1.5 hours before it was transferred to an oven at 37° C. and the drying continued overnight.

The resulting dry foams were soft and flexible with an open pore network. The pores in the alginate foam part were smaller than in the foam made from chitosan. It was not possible to separate the two foam types after drying. Each foam layers absorbed water instantly (the absorption time of the first added drop was less than 1 second for the chitosan foam and about 3 seconds for the alginate foam) and they remained attached after hydration. The hydrated alginate part of the hydrated foam had a high tensile strength whereas the hydrated chitosan part was very weak. Pieces of the hydrated chitosan foam broke off when a finger was pushed against the chitosan foam side or when the chitosan foam was stretched by pushing against the reverse (alginate foam) side. The failure was not delamination.

### EXAMPLE 12

This example describes a method for cross-linking a chitosan foam for making it more stable related to biodegradation and providing higher wet integrity.

A chitosan foam was made as described in example 11 except that the mixing times were 1.5 minutes and 4.5 minutes at medium and high speed respectively. The resulting wet foam density was 0.20 g/ml. The wet foam was cast in 2 mm and 4 mm deep molds. Then a 100 mM solution of Na-triphosphate filled in a spray bottle with the nozzle adjusted to give fine droplets. The Na-triphosphate solution was sprayed onto the wet foams about 50 ml and 100 ml for the 2 mm and the 4 mm respectively. The wet foams absorbed some of the solution sprayed on, so the addition was performed several times with less than a minute between each addition. The wet foams were then dried in a drying oven at 80° C. for 1 hour and 2 hours for the foams cast in the 2 mm and 4 mm molds respectively.

The dry foams were soft, flexible and had an open pore network. The foams absorbed water instantly and they deformed less upon hydration and were stronger than the non-crosslinked chitosan foams in Example 10.

### EXAMPLE 13

This example shows that a chitosan foam containing gelling ions will have the ability to induce gelling of an externally added chitosan solution in situ.

Foam disks (diameter=2.1 cm) were stamped out with use of a cork borer from the foam cast in the 4 mm high mold presented in Example 12. A foam disk was then placed on the serrated plate on the same rheometer as used in previous example. The disk was then added excess solution of either MQ-water or a 1.0% solution of chitosan (PROTASAN UP CL 213). The upper plate (PP25) was lowered to a gap of 500 μm and a stress sweep was performed with an applied shear stress from 0.5 Pa to 50 Pa. The oscillation measurements were initiated about three minutes after addition of solution. The frequency was set to 1 Hz. The sweep was performed two times for each foam patch. The elastic modulus, G′, read in the linear viscoelastic region (G′ₗᵢₙ) and the phase angle are reported in Table 18.

TABLE 18
G′ₗᵢₙ and phase angle measured for cross linked
chitosan foams added water and chitosan solution.

| Solution added | G′ₗᵢₙ ± SD, [Pa] | Phase angle, [°] |
|---|---|---|
| MQ-water | 502 ± 65 | 24.6 ± 0.3 |
| 1.0% chitosan solution | 777 ± 29 | 17.6 ± 4.1 |

Based on both the elastic modulus and the phase angle indicate the results in the table a more gel like properties of the foam after addition of chitosan solution.

### EXAMPLE 14

This example shows how the mixing time and amount of air incorporated into the chitosan foams affects different foam properties.

Chitosan foams were prepared as described in Example 10 except different mixing times were used to obtain different foam densities. All foam ingredients for creating a wet foam was mixed at medium speed for 1.5 minutes. Then mixing at high speed was continued for 1 minute with a resulting wet density 0.45 g/ml. About half of the foam was cast in 4 mm and 2 mm high molds. Then the remaining foam was mixed at high speed for one additional minute. The resulting wet density was 0.29 g/ml and the rest of the foam was cast as above. A similar procedure as above was repeated except for the mixing times at high speed were first 45 seconds and the second 4 minutes and 45 seconds. The wet densities were 0.52 g/ml and 0.18 g/ml respectively. The two foams with highest wet densities got a thin film created at the surface against the mold. This is due to coalescence of the pores as the foam dries more slowly near the bottom. The dry foam density was determined by stamping out disks, from the foam cast in the 4 mm high mold, with a diameter of 1 cm with use of a cork borer and weighing them. The densities and thickness measured by a caliper of the different foams are presented in table 18. The foams were also characterized by its elastic modulus, G in, with the same rheometer settings as described in Example 10 except the range of applied stress was 0.5 Pa to 18 Pa, and that three sweeps for each foam piece were performed. The results are included in table 19, presenting the average values of the two last sweeps for three different foams with a diameter of 1 cm. The foam pieces were kept in 2 ml Hanks' solution about five minutes before they were transferred to the rheometer. The tensile strength of the dried foams was measured with use of a SMS Texture Analyzer and A/TG tensile grips. The force required stretching the foam at 0.5 mm/s until breakage was read and maximum force and distance stretched when it ruptured are reported in table 19. The foam pieces were bone-shaped cut with use of a scalpel with the dimensions; 3.15 cm long, 1.75 cm wide at the ends and 1.25 cm wide in the center, the narrowing start 1 cm from the ends. The foam was cut in this shape to ensure breakage in the middle of the foam and not where it was attached to the grips. Approximate 0.3 cm of each end of the foam piece was used to fasten it to the grips.

TABLE 19
Chitosan foams of different density and their properties.
(n = 3, ±SEM) (The foam with wet density of 0.23 g/ml
is the foam from Example 10)

| Foam | | | | Distance | |
|---|---|---|---|---|---|
| wet | Foam dry | | | before | |
| density, | density, | Thickness, | Tensile | rupture, | |
| [g/ml] | [mg/cm²] | [mm] | strength, [g] | [mm] | G′ₗᵢₙ, [Pa] |
| 0.52 | 24.8 ± 0.2 | 2.4 | 138 ± 10 | 20 ± 2 | 133 ± 23 |
| 0.45 | 22.4 ± 0.7 | 2.5 | 148 ± 8 | 14 ± 2 | 115 ± 6 |
| 0.29 | 17.4 ± 0.4 | 3.2 | 79 ± 1 | 5.1 ± 0.2 | 55 ± 3 |
| 0.23 | 15.4 ± 0.2 | 3.4 | 60 ± 1 | 6.6 ± 0.3 | 51 ± 1 |
| 0.18 | 12.5 ± 0.3 | 3.7 | 49 ± 1 | 6.7 ± 0.5 | 9 ± 1 |

The table shows that the foams with the highest wet densities collapsed most due to coalescence. It was also observed that the foams had increasing pore size by increasing wet density. The tensile strength and the elastic modulus decreased by increased amounts of air. Also the elasticity of the materials presented as the length the material could be stretched before it ruptured decreased by decreasing wet density. The three less dense materials had about the same elasticity.

### EXAMPLE 15

This example describes the preparation of a hyaluronic acid (HA) foam with calcium ions incorporated. Also the foams ability to donate these ions to induce gelling of an externally added alginate solution is shown.

An aqueous solution containing 2.5% HA was prepared and set aside. 49.65 g MQ-water, 2.35 g CaCl₂*2H₂O and 10.5 g sorbitol (dry) were added a mixing bowl and the dry ingredients were dissolved by gently swirling the bowl. 130 g of the HA solution, 4.5 g glycerin and 3.0 g HPMC were added to the same mixing bowl. The dispersion was then blended with a Hobart kitchen aid mixer equipped with a wire whisk at medium speed for two minutes to ensure homogeneity. The mixing continued at high speed for 3 minutes and 50 seconds. The wet density was measured to be 0.21 g/ml (determined from the weight of wet foam required to fill a 100 ml container). The wet foam was cast in 2 mm and 4 mm high molds coated with Teflon and then placed in a drying oven at 80° C. for 50 minutes.

With use of a cork borer foam disks (diameter=2.1 cm) were stamped out from the foam cast in the 4 mm high mold. A 1.0% and 0.5% alginate solution was prepared from PRONOVA SLG 20 (batch: 221105) by addition of MQ-water. A dry foam disk was placed on a Bohlin CVO 120 High Resolution Rheometer between serrated plates (PP25). Then 350 μl of the alginate solution was added with use of a pipette. The calcium content in the foam disk is enough to saturate gelling residues of the added alginate by 124% and 248% for the 1.0% and 0.5% solution respectively. After one minute the alginate solution is absorbed and the foam is close to be fully hydrated. The upper plate was then lowered to 500 μm gap and measurements of the elastic modulus, G′, was initiated. The frequency, strain and temperature were set to 1 Hz, 0.001 and 20° C. respectively. The results are presented in table 20.

TABLE 20
Elastic modulus, G′, as a function of time after addition of water
and solutions to the HA foam with calcium ions incorporated.

| | Elastic modulus, G′, [Pa] | | |
|---|---|---|---|
| Time, [min] | MQ-water | 0.5% alginate | 1.0% alginate |
| 2 | 26 | 743 | 1665 |
| 4 | 31 | 660 | 2153 |
| 6 | 27 | 698 | 2544 |
| 8 | 25 | 750 | 3003 |
| 10 | 25 | 816 | 3322 |
| 15 | — | 1003 | 4167 |
| 20 | — | 1193 | 4732 |
| 25 | — | 1355 | 5608 |
| 30 | — | 1591 | 6292 |
| 35 | — | 1867 | 6602 |

The increase of G′ during the minutes just after addition of the alginate solution, confirms donation of gelling ions and that a gelling reaction have been initiated. The difference in G′ value between the three solutions confirms that a gel is being created and that the strongest gel is created from the most concentrated alginate solution.

This example describes the preparation of a HA foam with phosphate ions incorporated. Also the foams ability to donate these ions to induce gelling of an externally added chitosan solution is shown.

### EXAMPLE 16

This example describes the preparation of a HA foam with phosphate ions incorporated. Also the foams ability to donate these ions to induce gelling of an externally added chitosan solution is shown.

The HA foam was made as described in Example 15, except that the calcium source was replaced with 2.27 g Na₂HPO₄ and the amount of water used was 49.7 g. The mixing time at high speed was 3 minutes with gave a wet density of 0.17 g/ml. The foams cast in 2 mm and 4 mm molds were kept in the drying oven at 80° C. for 45 min and 75 min respectively.

The same parameters for rheological measurements as described in Example A were used. Water and 1.0% chitosan solution was added in excess amount. The values describing the elastic modulus, G′, and phase angle flattened off at the values presented in Table 21.

TABLE 21
Elastic modulus, G′, and phase angle of rehydrated
HA foams with phosphate ions incorporated.

| | Elastic modulus, | Phase angle, |
|---|---|---|
| Solution added | G′, [Pa] | [°] |
| 1% chitosan solution | 76 | 22 |
| MQ-water | 18 | 46 |

The results indicate that the foam added chitosan solution gets a more gel like behavior and is stiffer than the foam added MQ-water.

### EXAMPLE 17

This example describes the preparation of a chitosan foam that contains calcium ions. The calcium immobilized in the chitosan foam induced in situ gelling of an alginate solution when it was absorbed by the dry chitosan foam. Such structures may be useful in biomedical applications for cell immobilization or to provide controlled release of immobilized drugs, enzymes, hormones, etc.

A chitosan foam was made comprising the same amounts and ingredients as in example 11 except that 2.35 g of MQ-water were replaced with 2.35 g CaCl₂*2H₂O (80 mM). A wet foam with a wet density of 0.20 g/ml were made by mixing at medium and high speed for 1.5 minutes and 6 minutes respectively. The wet foam was cast in 2 mm and 4 mm high molds as described earlier. Then they were placed in a drying oven at 80° C. for 1.5 hours. The dry foams were soft and flexible with an open pore network and a dry density of 0.039±0.001 g/cm³. The foam absorbed water instantly and had wet integrity similar to the foams of same thickness in Example 10. This foam expanded less when Hanks' solution was added this foam compared with the foams from Example 10. The absorption capacity of Hanks' solution for this foam was measured to be 16.8±1.9 g/g foam (average value of three samples±SD). The pores of this foam were somewhat larger than the 4 mm thick foam from Example 10, this may be described by more coalescence due to decreased viscosity of the chitosan because of the ionic strength of the solution.

Foam discs, from the foam molded in 4 mm high trays, were stamped out with use of a cork borer with a diameter of 2.1 cm. A dry foam disc was the placed on a Bohlin CVO 120 High Resolution Rheometer between serrated plates (PP25). Then 500 μl of a 1% alginate (PRONOVA UP LVG) solution was added with use of a pipette. The calcium content in the foam disc is enough to saturate gelling residues of the added alginate by 96%. After one minute the alginate solution is absorbed and the foam is close to be fully hydrated. The upper plate was lowered to 1.000 mm gap and measurements of the elastic modulus, G′, were initiated. The frequency, strain and temperature were set to 1 Hz, 0.001 and 20° C. respectively. The results are presented in table 22.

TABLE 22
The elastic modulus, G′, as a function of time for chitosan
foams added alginate solution and water (n = 3).

| | Elastic modulus, G′ | Elastic modulus, G′, ± |
|---|---|---|
| Time, [min] | ± SD [Pa] (alginate) | SD [Pa] (water) |
| 1 | 4987 ± 5 | 470 ± 16 |
| 2 | 5867 ± 40 | 501 ± 13 |
| 3 | 6346 ± 15 | 516 ± 15 |
| 4 | 6653 ± 72 | 515 ± 9 |
| 5 | 6850 ± 64 | 529 ± 17 |
| 7 | 7078 ± 65 | 531 ± 23 |
| 9 | 7191 ± 76 | 532 ± 23 |
| 11 | 7216 ± 122 | 536 ± 21 |
| 12 | 7260 ± 120 | 534 ± 21 |

The high value of G′ for the foam discs added alginate solution and the increase in G′ during the minutes just after addition, confirms donation of gel-forming ions to the added alginate solution from the chitosan foam.

### EXAMPLE 18

### Prophetic Example

Following the procedure of Examples 2-9, 13, and 16, composites without the coating are prepared. Next, 1 mL (per cm² of foam) of a 5% w/v solution of Pronova SLM₂₀ is placed over one surface of the foam composite and the applied solution is contacted with a 100 mM solution of CaCl₂ in MOPS 1× washing buffer solution (Inotech Encapsulation AG, Dottikon, Switzerland) for 5 minutes. The composite is then washed twice with a calcium-free MOPS 1× buffer during 4 minutes. The other side of the composite is then coated by placing 1 mL (per cm² of foam) of a 5% w/v solution of Pronova SLM₂₀ over the opposite surface of the foam and then contacting the applied solution with a 100 mM solution of CaCl₂ in MOPS 1× washing buffer solution for 5 minutes. The composite is then washed twice with a calcium-free MOPS 1× buffer during 4 minutes.

### EXAMPLE 19

### Prophetic Example

Pancreas tails from adult pig donors are digested by a modified static digestion method as described by et al., “The isolation and function of porcine islets from market weight pigs,” Cell Transplant. 2001, 10:235-246, which is hereby incorporated by reference in its entirety. The pancreas are infused with a 2 to 3-fold volume (ml/gr) of Liberase PI (Roche/Boerhinger Mannheim, 0.5 mg/ml) dissolved in modified UW-M solution. The pancreas is injected in order to achieve an adequate distension, placed in a sterile 1 L Nalgene jar and digested by static incubation at 37° C. for 50 min. Digestion is terminated by addition of Ham-F10+20% NCS based on the visual inspection of the gland. The cell suspension is filtered through a stainless steel mesh with a pore size of 1000 μm and diluted in Ham-F10+20% NCS. Following previous data obtained in human islet isolation, digested tissue is passed over a bed of 6-mm glass beads and through a 500 stainless-steel mesh screen. The tissue effluent is collected with 3 to 4 L of cold Ham-F10+10% NCS in 250 ml conical tubes and centrifuged at 700 rpm at 4° C. Islets, cells and debris collected after the pre-purification column (8 tubes on average) is then centrifuged at 4° C. (630 g for 3 minutes). All cellular pellets are pooled in one tube and suspended in 200 ml Ham-F10 medium. From this suspension, 100 μl aliquots are taken to evaluate the results of the digestion after dithizone staining (see Isolation outcome). Cells are then centrifuged at 4° C. (280 g for 5 min), the supernatant is removed and cells are suspended in 75 ml Ficoll Eurocollins (Mediatech, Hemdon, USA) solutions for pl˜rification in gradient tubes (ref: nalg3122-0250; VWR International, Leuven, Belgium).

Islets isolated with the static method are purified, at 4° C. on a discontinuous Ficoll Euro-Collins gradient. The post-digestion cellular pellet, suspended in 75 ml of Ficoll Euro-Collins solution (density=1.1 g/cm3), are placed in a flat-bottom tube. Lower gradients of Ficoll are then added sequentially (50 ml of 1,096 g/cm3; 50 ml of 1,060 g/cm3 and 20 ml of Ham-F10 medium). After centrifugation of the gradient tubes at 856 g for 17 min, islets are collected from the 1.1/1.096 and 1.096/1.060 interfaces. Islets from each interface are suspended in 2 tubes containing 50 ml Ham-F10+10% NCS serum. The tubes are centrifuged at 280 g for 3 min, the supernatant is removed and the cells are washed with 150 ml Ham-F10 medium. This procedure is repeated 3 times and, finally, the islets are suspended in 200 ln1 Ham-F10 medium for isolation outcome study.

Following the procedure of Examples 6, foam disks (diameter=2.1 cm) are prepared. The foam discs are transferred to wells in a 12 well plate (Nunclon®, Nalgene Nunc International), where they closely fit the well size. 300 μl of a 0.5% w/v NOVATACH RGD solution containing 30,000 adult pig pancreatic cells is distributed drop-wise with a pipette to the foam. The foams are then incubated for 20 minutes at 37° C.

Next, a solution of 5% w/v solution of PRONOVA SLM₂₀ or a 3% solution of PRONOVA SLM₁₀₀ alginate matrix is placed over one surface of the foam composite using a 1 mL syringe and the applied solution is contacted with a 100 mM solution of CaCl₂ in MOPS 1× washing buffer solution (Inotech Encapsulation AG, Dottikon, Switzerland) for 5 minutes. The composite is then washed twice with a calcium-free MOPS 1× buffer during 4 minutes. After cultivation, the other side of the foam is then coated by placing a solution of 1% w/v PRONOVA SLM₂₀ or PRONOVA SLM₁₀₀ using a 1 mL syringe on the opposite side and then contacting the applied solution with a 100 mM solution of CaCl₂ in MOPS 1× washing buffer solution for 5 minutes. The composite is then washed twice with a calcium-free MOPS 1× buffer during 4 minutes.

### EXAMPLE 20

### Prophetic Example

Alginate foams are manufactured by mechanically agitating gelling agent dispersions (e.g. CaCO₃) of an aqueous solution of alginate, plasticizer, foaming agent and a slowly hydrolyzing acid. As a result of the gradual reduction in pH, calcium ions are released from the particles thereby inducing a controlled cross-linking of the alginate foam structure. The wet alginate foam is after agitation cast into a flat mould containing a polyester mesh. After gelling is completed the foam containing the mesh is air dried in an oven at 35-80° C. The foam is then cut into circles or squares with an area of about 1 cm² and placed in a sealed package before being sterilized by autoclaving or irradiation.

The day before transplantation islets are purified from donor animals or humans and carefully applied to the foam in a Petri dish as a suspension containing 0.8% NOVATACH RGD using a pipette. This allows the islets to be entrapped in the foam-gel formed structure. After a few minutes, a selected alginate with a high content of mannuronic acid (PRONOVA SLM₂₀) in a 5% concentration is carefully applied as a few droplets in a Petri dish. The foam containing the islets is placed on top of the viscous alginate solution and a few more droplets are thereafter added on the upper side of the foam structure until it is completely coated. A 100 mM CaCl₂ solution in MOPS buffer is then carefully filled into the Petri-dish until the foam-gel is completely covered with the solution, thereby completely saturating the gel-structure with gelling ions. After about 5 minutes the CaCl₂ solution is replaced by a culture media adapted for islets containing 5 mM CaCl₂ and 5 mM SrCl₂. This allows the foam-gel structure to further rearrange and stabilize. After about 2 hours the medium is again changed to a cell medium without additional calcium or strontium. The device is stored in a CO₂ incubator until a subcutaneous implantation is performed in diabetic primates on the following day. The device is designed to regulate body insulin for several months and may later be replaced by a similar device.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

### EXAMPLE 21

The alginate used in this example was added calcium sufficient to saturate 75% of the alginate. The wet foam formulation is presented in Table 23.

TABLE 23
Wet foam formulation.

| Ingredient | Amount, [g] |
|---|---|
| 4% alginate solution | 111.2 |
| (PRONOVA UP MVG, 701-256-11) | |
| Glycerin | 6.0 |
| Sorbitol (dry) | 14.0 |
| HPMC | 3.0 |
| CaCO₃ (HuberCal 500 Elite) | 0.25 |
| CaCO₃ (HuberCal 250 Elite) | 0.26 |
| GDL | 1.81 |
| MQ-water | 63.5 |
| Total | 200.0 |

The foam was made as described in Example 1, except that the mixing time at high speed was 6 minutes before addition of the GDL solution and 1.5 minutes after. The resulting wet density of the foam was 0.22 g/ml. The foam was then cast on a 2 mm high mold coated with Teflon. Then a circular macroporous filter (Spectra/Mesh macroporous filter, diameter: 55 mm, mesh opening: 300 μm, open area: 44%, thickness: 258 μm, art.: 148 300 from Spectrum Laboratories Inc., Rancho Dominguez, Calif., USA) was placed on top of the foam and the height of the mold was increased by 2 mm by adding an additional frame and a layer of wet foam was added on top of the filter. The layered material was then kept uncovered at ambient temperature for 1 hour during gelling before it was dried at 80° C. for 1 hour. Alternatively, the wet foam was cast either on top of a Teflon coated mold (2 mm high) both with and without a filter placed between the Teflon and the foam. The foams were then gelled for 1 hour before they were layered resulting in a foam with the filter in the middle. The foams were then dried at 80° C. for 1 hour. Both of the dried foams appeared similar, but the first described method is preferred because this was the easiest.

SiLi beads® (type: ZY Premium (sintered Zirconia beads, yttrium stabilized), 0.1 mm-0.2 mm, art.: 97015, Sigmund Lindner GmbH, Warmensteinach, Germany) was then suspended in a 0.6% alginate solution (PRONOVA UP LVG, FP-502-04) and dripped onto the foam with use of a pipette. After 10 minutes was the foam investigated with use of a light microscope and the beads were seen distributed through the foam at different levels. SiLi beads were selected to determine whether particles of a similar size to the size of some cells would be distributed within the foam when added as a suspension in an alginate solution that was then dripped onto the foam.

What is claimed is:

1. A composite comprising:
i) a foam having pores, wherein said foam comprises a polymer and gel-forming ions distributed through at least a part of the foam;
ii) a gel comprising a polysaccharide with gelling sites, wherein the gel is formed and located within the pores of the foam and interacts with the foam; and
iii) a coating comprising a polysaccharide with gelling sites and gel-forming ions, wherein said coating completely covers the outer surface of said foam.

2. A composite according to claim 1, wherein said polymer is selected from the group consisting of an alginate, a pectin, a carrageenan, a hyaluronate, chitosan, a cell adhesion sequence modified alginate, a RGD modified alginate and combination thereof.

3. A composite according to claim 1, wherein: said gel-forming ions in said foam are selected from the group consisting of strontium ions, barium ions, calcium ions, and combination thereof; and said polysaccharide is selected from the group consisting of an alginate, a pectin, a carrageenan, a hyaluronate, and combination thereof; or said gel-forming ions in said foam and said coating are each independently selected from the group consisting of triphosphate ions, phosphate ions, citrate ions, or combination thereof; and said polysaccharide comprises a chitosan.

4. A composite according to claim 1, wherein said gel-forming ions in said foam are present in a molar amount equivalent to at least 5% of said gelling sites of said polysaccharide of said gel.

5. A composite according to claim 1, wherein said polysaccharide in said gel is selected from the group consisting of an alginate, a chitosan, a carrageenan, a hyaluronate, a pectin, a cell adhesion sequence modified alginate, a RGD modified alginate and combination thereof and said coating comprises a hyaluronate, alginate, chitosan, a cell adhesion sequence modified polysaccharide or a RGD modified alginate.

6. A composite according to claim 1, wherein a polysaccharide comprises an alginate with a guluronate (G) content of about 20% to about 80% and/or an alginate with a mannuaronate (M) content of at least 20%, and/or a mannuaronate to guluronate ratio equal to or greater than about 1.

7. A composite according to claim 1, wherein said polysaccharide comprises an alginate with a weight-average molecular weight selected from the group consisting of about 4 kD to about 300 kD, about 150 kD to about 250 kD and about 75 kD to about 150 kD.

8. A composite according to claim 1, wherein said polysaccharide in said coating is present in a range selected from the group consisting of: about 0.2% to about 10% of said coating; about 1% to about 8% of said coating; about 2% to about 7% of said coating; and about 3% to about 6% of said coating.

9. A composite according to claim 1, wherein the foam: absorbs from 1 to 30 times its weight of a liquid selected from the group water, a physiological solution, a soluble polysaccharide solution, and a polysaccharide solution comprising a functional component; or comprises an alginate, the composite further comprising a functional component selected from a pharmaceutical active and/or an anti-adhesive agent; and/or further comprises a mesh.

10. A composite according to claim 1, wherein the elastic modulus of the composite is from 0.1 kPa to 1000 kPa.

11. A composite according to claim 1, further comprising a functional component comprising a flavor, a fragrance, a particulate, cells, multicellular aggregates, a therapeutic agent, a tissue-regenerative agent, a growth factor, a nutraceutical, and combination thereof.

12. A composite according to claim 1, wherein said gel further comprises pancreatic islet cells, hepatic cells, neural cells, vascular endothelial cells, parathyroid cells, thyroid cells, adrenal cells, thymic cells, mesenchymal stem cells and ovarian cells.

13. A composite according to claim 1 optionally comprising cells, said composite selected from the group consisting of:
a) a composite wherein:
i) said polymer in said foam is an alginate, cell adhesion sequence modified alginate, and combination thereof;
ii) said gel-forming ions in said foam are selected from the group consisting of calcium ions, strontium ions, barium ions, and combination thereof;
iii) said polysaccharide in said gel is selected from the group consisting of an alginate, cell adhesion sequence modified alginate, and combination thereof; and
iv) said gel-forming ions in said coating are selected from the group consisting of calcium ions, strontium ions, barium ions, and combination thereof;
b) a composite wherein:
i) said polymer in said foam is an alginate;
ii) said gel-forming ions in said foam are selected from the group consisting of calcium ions, strontium ions, barium ions, and combination thereof;
iii) said polysaccharide in said gel is an alginate;
iv) said coating comprises an alginate; and
v) said gel-forming ions in said coating are selected from the group consisting of calcium ions, strontium ions, barium ions, and combination thereof;
c) a composite wherein:
i) said polymer in said foam is an alginate;
ii) said gel-forming ions in said foam are selected from the group consisting of calcium ions, strontium ions, barium ions, and combination thereof;
iii) said polysaccharide in said gel is a RGD modified alginate;
iv) said coating comprises an alginate; and
v) said gel-forming ions in said coating are selected from the group consisting of calcium ions, strontium ions, barium ions, and combination thereof;
d) a composite wherein:
i) said polymer in said foam is an alginate;
ii) said gel-forming ions in said foam are selected from the group consisting of calcium ions, strontium ions, and combination thereof;
iii) said polysaccharide in said gel is a RGD modified alginate;
iv) said coating comprises an alginate; and
v) said gel-forming ions in said coating are selected from the group consisting of calcium ions, strontium ions, and combination thereof; and
e) a composite wherein:
i) said polymer in said foam is an alginate;
ii) said gel-forming ions in said foam are calcium ions;
iii) said polysaccharide in said gel is a RGD modified alginate;
iv) said coating comprises an alginate; and
v) said gel-forming ions in said coating are calcium ions.

14. A composite according to claim 1, wherein the average pore size of said pores is about 25 μm to about 1000 μm in diameter.

15. A composite according to claim 1, further comprising a solid support structure.

16. A composite according to claim 15, wherein the foam comprises a solid support structure that is a mesh.

17. A method of forming the composite of claim 1, comprising:
i) contacting a first liquid component with a foam having pores, wherein said first liquid component comprises a polysaccharide having gelling sites capable of forming a gel upon contact with said ions, whereby said gel forms upon contact with the said gel-forming ions; and
ii) forming said coating on the outer surface of said foam; and
iii) optionally contacting said composite with a solution of strontium ions and calcium ions after said forming of said coating;
wherein:
said foam comprises gel-forming ions, wherein said ions are distributed through at least a part of the foam; and
said coating comprises polysaccharide with gelling sites and gel-forming ions, wherein said coating completely covers the outer surface of said foam.

18. A method according to claim 17, wherein said forming of said coating comprises:
i) applying a second liquid component comprises a polysaccharide with gelling sites to the outer surface of the foam; and
ii) after said applying, contacting said second liquid component with a solution of gel-forming ions to form said coating.

19. A method according to claim 17, wherein said first liquid component comprises: about 0.2% w/v to about 10% w/v of said polysaccharide; about 0.5% w/v to about 5% w/v of said polysaccharide; or about 0.1% w/v to about 1.0% w/v of said polysaccharide; and/or said second liquid component comprises about 0.2% w/v to about 10% w/v of an alginate, modified alginate, or combination thereof, or about 2% w/v to about 7% w/v of an alginate, modified alginate, or combination thereof.

20. A method for controlling cell proliferation in the composite defined in claim 1, comprising washing the composite with a solution, adjusted for osmotic pressure neutral to living cells, and which solution contains gel-forming ions.

21. A method for recovery of cells from the composite according to claim 1, comprising contacting an aqueous solution comprising a recovery agent with the gel comprising the polysaccharide; wherein said gel comprises cells; and said aqueous solution is adjusted to provide an osmotic pressure neutral to living cells.

22. Use of a composite according to claim 1, as a matrix for cell immobilization and/or proliferation for an in vitro tissue culture application and/or an in vivo tissue engineering application and/or as a composite further comprising an therapeutic agent to provide in vivo controlled release of the active agent into a human or animal body; and/or as a composite further comprising a component for use in managing a wound to provide in vivo wound management.

23. A method for preventing adhesion of tissue to adjacent tissue comprising applying to the tissue a composite according to claim 1, such that it provides a barrier between the tissue and the adjacent tissue.

24. A method of administering one or more composites to a patient in need thereof, comprising implanting said one or more composites according to claim 1, in said patient.

25. A method according to claim 24, wherein said gel comprises pancreatic islet cells and said patient is in need of treatment of diabetes or regulation of blood glucose levels.

26. Use of a composite according to claim 1, for use in a method of treatment of diabetes or a method of reducing blood glucose levels.


SEQUENCE LISTING (22 sequences)

SEQ ID NO: 1 - protein, 5 aa, Artificial Sequence
  Cell Attachment Peptide 1
YIGSR

SEQ ID NO: 2 - protein, 5 aa, Artificial Sequence
  Cell Attachment Peptide 2
IKVAV

SEQ ID NO: 3 - protein, 4 aa, Artificial Sequence
  Cell Attachment Peptide 3
REDV

SEQ ID NO: 4 - protein, 4 aa, Artificial Sequence
  Cell Attachment Peptide 4
DGEA

SEQ ID NO: 5 - protein, 6 aa, Artificial Sequence
  Cell Attachment Peptide 5
VGVAPG

SEQ ID NO: 6 - protein, 5 aa, Artificial Sequence
  Cell Attachment Peptide 6
GRGDS

SEQ ID NO: 7 - protein, 4 aa, Artificial Sequence
  Cell Attachment Peptide 7
RGDV

SEQ ID NO: 8 - protein, 5 aa, Artificial Sequence
  Cell Attachment Peptide 8
PDSGR

SEQ ID NO: 9 - protein, 7 aa, Artificial Sequence
  Cell Attachment Peptide 9
RYVVLPR

SEQ ID NO: 10 - protein, 6 aa, Artificial Sequence
  Cell Attachment Peptide 10
LGTIPG

SEQ ID NO: 11 - protein, 7 aa, Artificial Sequence
  Cell Attachment Peptide 11
LAGRGDS

SEQ ID NO: 12 - protein, 4 aa, Artificial Sequence
  Cell Attachment Peptide 12
RGDF

SEQ ID NO: 13 - protein, 12 aa, Artificial Sequence
  Cell Attachment Peptide 13
HHLGGALQAGDV

SEQ ID NO: 14 - protein, 4 aa, Artificial Sequence
  Cell Attachment Peptide 14
VTCG

SEQ ID NO: 15 - protein, 4 aa, Artificial Sequence
  Cell Attachment Peptide 15
SDGD

SEQ ID NO: 16 - protein, 6 aa, Artificial Sequence
  Cell Attachment Peptide 16
GREDVY

SEQ ID NO: 17 - protein, 5 aa, Artificial Sequence
  Cell Attachment Peptide 17
GRGDY

SEQ ID NO: 18 - protein, 6 aa, Artificial Sequence
  Cell Attachment Peptide 18
GRGDSP

SEQ ID NO: 19 - protein, 4 aa, Artificial Sequence
  Cell Attachment Peptide 19
VAPG

SEQ ID NO: 20 - protein, 9 aa, Artificial Sequence
  Cell Attachment Peptide 20
GGGGRGDSP

SEQ ID NO: 21 - protein, 8 aa, Artificial Sequence
  Cell Attachment Peptide 21
GGGGRGDY

SEQ ID NO: 22 - protein, 6 aa, Artificial Sequence
  Cell Attachment Peptide 22
FTLCFD